(19) United States
(12) Patent Application Publication
(10) Pub. No.: US 2005/0042291 A1
(43) Pub. Date: Feb. 24, 2005

(54) Diffusion layer modulated solids

(76) Inventors: Michael Hawley, Kalamazoo, MI (US); Walter Morozowich, Kalamazoo, MI (US); Michael S. Bergren, Portage, MI (US); John W. Skoug, Portage, MI (US); Phillip R. Nixon, Portage, MI (US); John M. Heimlich, Portage, MI (US); Ping Gao, Portage, MI (US)

Correspondence Address: MUETING, RAASCH & GEBHARDT, P.A., P.O. BOX 581415, MINNEAPOLIS, MN, 55458, US

(21) Appl. No.: 10/881,712

(22) Filed: Jun. 29, 2004

Related U.S. Application Data
(60) Provisional application No. 60/484,205, filed on Jul. 1, 2003.

(30) Foreign Application Priority Data
Jun. 29, 2004 (WO) PCT/US04/21143

(51) Int. Cl. A61K 9/24

(52) U.S. Cl. 424/473

(57) ABSTRACT

Diffusion layer modulated solids that include an excipient and a soluble salt of a poorly soluble, basic drug; a soluble salt of a poorly soluble, acidic drug; or a poorly soluble, non-ionizable drug are useful, for example, for improved delivery of drugs.

This application claims the benefit of U.S. Provisional Application No. 60/484,205, filed Jul. 1, 2003, which is herein incorporated by reference in its entirety.

### BACKGROUND

Solubility is one of the most important factors in the design and development of drug formulations. For example, the oral bioavailability of a drug is often limited by the aqueous solubility of the drug. Soluble salts of poorly soluble acidic or basic drugs have been prepared in attempts to enhance the oral bioavailabilities of the drugs, and in some cases the oral bioavailabilities are improved. However, in a number of cases the oral bioavailability of the soluble salt of a poorly soluble drug is no higher than the oral bioavailability of the parent free acid or base, and in some cases the salt has an even lower oral bioavailability than that of the parent drug (e.g., sodium warfarin as compared to warfarin; sodium phenobarbital as compared to phenobarbital).

One of the reasons for the unpredictable dissolution and oral bioavailability behavior of drug salts has been attributed to the propensity of the salts of poorly soluble drugs to undergo dissociation or “salt hydrolysis” on contact of the drug salt with water, leading to the formation of the free acid or base, and subsequent precipitation of the corresponding free acid or free base form of the drug.

When the solution concentration of the resulting free acid or free base form of the drug greatly exceeds the solubility of the drug at the pH generated in the aqueous diffusion layer, precipitation of the poorly soluble, free acid or free base form of the drug may occur either directly on the surface of the dissolving drug salt, or at a site removed from the surface of the dissolving drug salt crystals. This can lead to a reduction in the dissolution rate, as well as a reduction in the oral bioavailability, of a soluble salt of a poorly soluble drug.

Salts of poorly soluble drugs may be formulated with simple physical mixtures of excipients that serve as diluents or vehicles for the drug, which can lead to increased solubility of the drug through alteration of the bulk solution pH. Useful excipients include neutral, acidic, and basic materials. In the case of salts of poorly soluble, basic drugs, it is known to use acidic materials as excipients to increase the solubility of the basic drug in solution through alteration of the pH of the bulk solution. Likewise, in the case of salts of poorly soluble, acidic drugs, it is known to use basic materials as excipients to increase the solubility of the basic drug in solution through alteration of the pH of the bulk solution. In addition, in the case of poorly soluble non-ionizable drugs, it is known to use solubilizing physical mixtures containing solubilizing excipients to increase the solubility of the drug in the bulk solution.

However, the use of these simple physical mixtures of soluble salts of poorly soluble, basic drugs with acidic excipients; soluble salts of poorly soluble, acidic drugs with basic excipients; and poorly soluble non-ionizable drugs with solubilizing excipients does not generally increase the rate of dissolution of the drug to levels that would lead to the desired improvement in oral absorption.

Poorly soluble drugs and/or their salts with enhanced dissolution rates, and methods of enhancing the rate of dissolution of poorly soluble drugs and/or their salts are needed in the art.

### SUMMARY OF THE INVENTION

In one aspect, the present invention provides diffusion layer modulated solids and methods of preparing diffusion layer modulated solids. Compositions, capsules, and tablets that include diffusion layer modulated solids are also provided.

In one embodiment, the diffusion layer modulated solid includes a soluble salt of a poorly soluble, basic drug and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

In another embodiment, the diffusion layer modulated solid includes a soluble salt of a poorly soluble, acidic drug and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

In another embodiment, the diffusion layer modulated solid includes a poorly soluble, non-ionizable drug and a solubilizing excipient; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

In another aspect, the present invention provides a diffusion layer modulated solid including particles. In one embodiment, the particles include a soluble salt of a poorly soluble, basic drug and an excipient selected from the group consisting of acidic excipients, solubilizing excipients, and combinations thereof. In another embodiment, the particles include a soluble salt of a poorly soluble, acidic drug and an excipient selected from the group consisting of basic excipients, solubilizing excipients, and combinations thereof. In another embodiment, the particles include a poorly soluble, non-ionizable drug and a solubilizing excipient.

Preferably, diffusion layer modulated solids provide for increased bioavailability of drugs, which may offer improved methods of treating diseases.

Definitions

As used herein, “drug” means a pharmacologically active compound.

As used herein, “poorly soluble drug” means a drug having a solubility of at most 50 micrograms/ml in an aqueous fluid at pH 6 to pH 7 at 25° C.

As used herein, “acidic drug” means a drug having a pKₐ of at most 11.

As used herein, “basic drug” means a drug having a pKₐ of at least 1.

As used herein, “soluble salt” means a drug having solubility of at least 50% greater than that of the non-salt form of the drug in an aqueous fluid at pH 6 to pH 7 at 25° C.

As used herein, the term “solid” is intended to encompass solid forms of matter including, for example, powders and compressed powders.

As used herein, “excipient” means a pharmaceutically inactive ingredient in a pharmaceutical formulation.

As used herein, “acidic excipient” means an excipient having a pKₐ of at most 6.

As used herein, “basic excipient” means an excipient having a pKₐ of at least 4.

As used here, “solubilizing excipient” means an excipient that results in increased drug solubility for a mixture of the drug and the excipient compared to the drug in the absence of the excipient.

As used herein, “intrinsic dissolution rate” refers the amount of drug dissolved per unit area per unit time.

As used herein, “crystal growth inhibitor” means a compound that slows the rate of crystal growth compared to the rate of growth without the crystal growth inhibitor.

As used herein, “particle” means a tiny mass of solid material.

As used herein, the term “granules” refers to a solid material consisting of a collection of particles adhered to one another.

As used herein, “granulating” means a process of increasing aggregate size by adhering particles together.

As used herein, “average size” refers to the average diameter of a group of particles. For non-spherical particles, the diameter is taken to be the longest dimension of the particle.

As used herein, “homogeneous” refers to a material of uniform composition. As used herein, “micronized” means a solid material that has been processed through a micronizer to reduce the average particle size.

As used herein, the term “tablet” refers to a solid, compressed form of a solid (e.g., drugs, drug salts, and/or excipients).

As used herein, the term “capsule” refers to a solid polymeric shell used for delivering its contents (e.g., drugs, drug salts, and/or excipents) to a desired site. Generally, the contents are release upon dissolution of the shell.

As used herein, “roller compaction” means a process of using a roller compactor to compress mixtures of materials (e.g., solids) at high pressures.

As used herein, “spray drying” means the process of expanding a liquid by forcing a high pressure liquid through a small diameter orifice into a drying chamber.

As used herein, “volatile liquid” means a liquid with a vapor pressure equal to or greater than the vapor pressure of water.

As used herein, “bioavailablity” means the AUC (area under the plot of plasma concentration of drug against time after drug administration) observed after oral administration divided by the AUC observed after IV administration multiplied by 100 to express the value as a percentage.

### BRIEF DESCRIPTION OF THE DRAWINGS

FIG. 1 illustrates the chemical structures of drugs. FIG. 1a is an illustration of the chemical structure of a soluble salt (i.e., delavirdine mesylate) of a poorly soluble, basic drug (i.e., delavirdine). FIG. 1b is an illustration of the chemical structure of a soluble salt (i.e., tipranavir disodium) of a poorly soluble, acidic drug (i.e., tipranavir). FIG. 1c is an illustration of the chemical structure of a poorly soluble, basic drug. FIG. 1d is an illustration of the chemical structure of the soluble hydrochloride salt of a poorly soluble, basic drug. FIG. 1e is an illustration of the chemical structure of a poorly soluble, non-ionizable drug. FIG. 1f is an illustration of the chemical structure of a poorly soluble, acidic drug.

FIG. 2 is a graph showing the intrinsic dissolution rate profile (x-axis is time in minutes, y-axis is concentration in micrograms/ml for delavirdine mesylate-citric acid (2:1) admixture co-compressed (Carver press) at pH 6 with 0.6% SLS. Also shown is the intrinsic dissolution rate profile for delavirdine mesylate alone at pH 2 and at pH 6 with 0.6% SLS at 37° C. The delavirdine mesylate-citric acid co-compressed admixture is approximately 100% dissolved in less than 10 minutes at pH 2 and pH 6. Delavirdine mesylate alone is only approximately 2% dissolved in 60 minutes at pH 6 with 0.6% SLS, and at pH 2, only approximately 60% dissolution occurs.

FIG. 3 illustrates a plot showing the effect of pH on the pellet intrinsic dissolution rate (micrograms·cm⁻²·second⁻¹) of delavirdine mesylate alone and a delavirdine mesylate-citric acid (2:1) co-compressed admixture along with the theoretical dissolution rate of delavirdine mesylate. The dissolution of a highly water soluble salt such as delavirdine mesylate should have very little pH dependency. However, the bulk drug alone has a very strong dependency on the bulk pH due to surface precipitation of a free base layer at pH 6. The co-compression of citric acid with delavirdine mesylate prevents free base formation on the dissolving surface, which in turn results in a substantially increased dissolution rate at pH 6.

FIG. 4 is an illustration of an overlay of a select portion of the powder X-ray diffraction (XRD) patterns (x-axis is two theta angle, y-axis is counts per second) of the remains from a dissolution pellet study with delavirdine mesylate at pH 2 and the reference XRD spectra for delavirdine free base and Forms XI (anhydrous) and XIV (trihydrate) of delavirdine mesylate. The dissolution pellet was obtained from a 15 minute intrinsic dissolution rate study at pH 2.0 HCl, at 300 rpm and 37° C. and the X-ray spectra were recorded a few days later. The XRD spectum of the dissolution pellet shows the presence of crystalline anhydrous delavirdine free base and the dihydrate of delavirdine mesylate (Form XIV) in roughly similar amounts (see the region at 17°-18° two theta) along with non-crystalline material (possibly delavirdine free base) and a trace amount of delavirdine mesylate, Form XI salt.

FIG. 5 is a graphical illustration of the intrinsic dissolution rates (micrograms·cm²·second⁻¹) for delavirdine mesylate-citric acid granules at 37° C. The dissolution rate of the granules (left and center) was virtually pH independent, in marked contrast to the bulk drug, delavirdine mesylate (right). The presence of magnesium stearate in the granules reduced the dissolution rate significantly (lot JMH-004a, left vs. JMH-004b, center).

FIG. 6 is a graphical representation of the USP dissolution profile (x-axis is time in minutes, y-axis is percent dissolved) at pH 6 with 0.6% SLS for delavirdine mesylate-lactose granules and delavirdine mesylate-citric acid granules.

FIG. 7 is a graphical representation (x-axis is time in hours, y-axis is concentration in micrograms/ml) of rat average plasma levels of delavirdine after administration of the delavirdine mesylate-citric acid co-compressed granular admixture (squares) and a delavirdine mesylate tablet available under the trade designation RESCRIPTOR from Pfizer Inc., New York, N.Y. (circles), after oral administration to rats at a stomach pH of 5 and a dose of 20 mg/kg (n=4).

FIG. 8 is a graphical illustration (x-axis is time in hours, y-axis is concentration in micrograms/ml) of rat blood level curves after oral admisinstratoin of gelatin capsules containing: the diffusion layer modulated solid prepared from tipranavir disodium spray dried powder, THAM, and PVP with addition of sodium laruryl sulfate (▪); and bulk tipranavir disodium(♦). The dose was 20 mg/kg of tipranavir in both cases. All formulations were administered to groups of 7-8 rats by oral intubation. Plasma samples were assayed by high pressure liquid chromatography (HPLC). The composition of the formulations is given in Table 4 and the AUC_{Inf} values are given in Table 5.

FIG. 9 is a graphical illustration (x-axis is time in minutes, y-axis is concentration in micrograms/ml) of the pH dependence of the dissolution behavior of the soluble hydrochloride salt of the poorly soluble, basic drug illustrated in FIG. 1c. The dissolution rate drops off sharply as the pH is increased despite the fact that the solubility of the salt is relatively constant over this range.

FIG. 10 is a graphical illustration (x-axis is time in minutes, y-axis is concentration in micrograms/ml) of the dissolution profile for a soluble hydrochloride salt of the poorly soluble, basic drug illustrated in FIG. 1c co-compressed with an acidic excipient, citric acid. The dissolution of the co-compressed material was far more rapid than that of the salt alone at pH 4.

FIG. 11 is a graphical illustration (x-axis is time in hours, y-axis is concentration in nM/ml) of plasma concentration of the poorly soluble, basic drug illustrated in FIG. 1c vs. time for individual subjects after administration of the drug. FIG. 11a depicts the administration of the HCl-salt of the poorly soluble, basic drug illustrated in FIG. 1c. The 24 hour points for subject 1 and 2 were not included in calculation of pharmacokinetic characteristics. FIG. 11b. depicts the administration of a pH-modulated solid including the hydrochloride salt of the poorly soluble, basic drug illustrated in FIG. 1c co-compressed with citric acid.

FIG. 12 depicts the dissolution profiles (x-axis is time in minutes, y-axis is concentration in micrograms/ml) for mixtures of a soluble salt (e.g., delavirdine mesylate) of a poorly soluble, basic drug (e.g., delavirdine) with an acidic excipient (e.g., citric acid) as a function of compression. FIG. 12a illustrates powder dissolution data at pH 6 (0.05M phosphate) for a 2:1 (w/w) mixture of delavirdine mesylate:citric acid. Dissolution of the co-compressed powder is far more rapid than the hand ground mixture of the two excipients. FIG. 12b illustrates a dissolution profile for a co-compressed diffusion layer modulated solid (SB) as compared to a hand ground mixture of the components (5A) in a dissolution basket at pH 6 and 25° C. The diffusion layer modulated solid was made from delavirdine mesylate:citric acid:lactose (2:1:1 w/w/w). Sample 5A was hand ground and placed as a powder in a dissolution basket. Sample 5B was co-compressed, then hand ground and placed as a powder in a dissolution basket. The diffusion layer modulated solid exhibits more rapid dissolution and also shows the ability to generate a solution of higher concentration than the mixture of the components alone.

FIG. 13 illustrates relative dissolution rates of 1:1 delavirdine mesylate:citric acid mixtures (w:w) dissolving in a capsule in pH 6 media as a function of compression of the mixtures. Dissolution rates were determined as the initial slope of the drug concentration vs. time profiles obtained after dissolution began.

FIG. 14 illustrates the dissolution profile (x-axis is time in minutes, y-axis is sample dissolved in mg) for mixtures of the soluble hydrochloride salt (i.e., illustrated in FIG. 1d) of a poorly soluble, basic drug with an acidic excipient (e.g., malic acid) using a rotating disk procedure for dissolution at pH 6 and 25° C. for co-compressed mixtures of the soluble hydrochloride salt illustrated in FIG. 1d with various weight fractions (0-40%) of malic acid. Significant enhancement in the dissolution rate was observed even at as low as 7% by weight malic acid.

FIG. 15 illustrates dissolution profiles (x-axis is time in minutes, y-axis is sample dissolved in mg) for co-compressed mixtures of the soluble hydrochloride salt (i.e., illustrated in FIG. 1d) of a poorly soluble, basic drug with acidic excipients (e.g., citric acid, malic acid, fumaric acid, xinatoic acid, and aspartame) using a rotating disk procedure for dissolution at pH 6 and 25° C. All sample were prepared with equivalent mole ratios (approximately 1:1). The highest dissolution rates were observed using fumaric acid, malic acid, and citric acid as the acidic excipient. The dissolution profile for the hydrochloride salt with no excipient is included for comparison.

FIG. 16 is a depiction of light microscopical examinations (7-400×) of samples of delavirdine mesylate:citric acid mixtures. FIGS. 16a and 16b represent samples prepared by roller compacted granulation and FIGS. 16c and 16d represent samples prepared by mortar and pestle. FIGS. 16a and 16c are at the same lower magnification, and FIGS. 16b and 16d are at the same higher magnification. The samples revealed significant differences in particle size and component distribution. Particle sizes of the sample produced by mortar and pestle were much smaller overall (FIGS. 16c and 16d) than the sample prepared by roller compacted granulation (FIGS. 16a and 16b).

FIG. 17 is an illustration of a Raman microscopy line map (x-axis is Raman shift in cm⁻¹, y-axis is counts) across a bisected granule prepared by roller compacted granulation of a mixture of delavirdine mesylate and citric acid.

FIG. 18 is an illustration of Raman spectra (x-axis is Raman shift in cm⁻¹, y-axis is counts) with the middle spectrum representing one point from the Raman line map across a bisected granule prepared by roller compacted granulation of a mixture of delavirdine mesylate and citric acid. The top spectrum represents delavirdine mesylate and the bottom spectrum represents citric acid.

FIG. 19 is an illustration of Raman spectra (x-axis is Raman shift in cm⁻¹, y-axis is counts) for typical individual crystals prepared from a mixture of delavirdine mesylate and citric produced by mortar and pestle (the middle two spectra), with the second from the top spectrum representing tan-brown pleochroic particles and the third from the top spectrum representing colorless particles. The top spectrum represents delavirdine mesylate and the bottom spectrum represents hydrous citric acid.

FIG. 20 is an illustration of an infrared microspectroscopy line map (x-axis is wavenumbers in cm⁻¹, y-axis is absorbance) of flattened granule prepared by roller compacted granulation of a mixture of delavirdine mesylate and citric acid with a spatial resolution of 15 micrometers.

FIG. 21 is an illustration of an infrared spectrum (x-axis is wavenumbers in cm⁻¹, y-axis is absorbance) of a typical point from the line map across a bisected granule prepared by roller compacted granulation of a mixture of delavirdine mesylate and citric acid (middle spectrum). The top spectrum represents hydrous citric acid and the bottom spectrum represents delavirdine mesylate.

FIG. 22 is a graph showing the intrinsic dissolution rate profile (x-axis is time in minutes, y-axis is concentration in micrograms/ml for the poorly soluble, non-ionizable drug illustrated in FIG. 1e-urea-sodium dodecyl sulfate (SDS) (66:33:1) admixture co-compressed (Carver press) ( [symbol] ) with 0.01N HCl at pH 2 as the dissolution media at 37° C. Also shown is the intrinsic dissolution rate profile for the poorly soluble, non-ionizable drug illustrated in FIG. 1e alone (○). The dissolution rate for the co-compressed the poorly soluble, non-ionizable drug illustrated in FIG. 1e-urea-SDS admixture was more than 100 times greater than that of the poorly soluble, non-ionizable drug illustrated in FIG. 1e alone in pH 2, 0.01N HCl at 37° C. The leveling off of the dissolution rate for the co-compressed admixture at after two minutes was due to the fact that the entire pellet had nearly dissolved at this point.

FIG. 23 is a graph showing the solubility of the poorly soluble, non-ionizable drug illustrated in FIG. 1e (y-axis is concentration of the poorly soluble, non-ionizable drug illustrated in FIG. 1e in mg/ml) in aqueous solutions of urea (x-axis is urea concentration in g/ml). The solubility of the poorly soluble, non-ionizable drug illustrated in FIG. 1e increased as the urea concentration increased.

FIG. 24 illustrates the dissolution profile (x-axis is time in minutes, y-axis is percent sample dissolved) for the free acid of the poorly soluble, acidic drug illustrated in FIG. 1(f) in capsules (-▴-); for the TRIS salt of the poorly soluble, acidic drug illustrated in FIG. 1(f) (-▪-); and for the TRIS salt of the poorly soluble, acidic drug illustrated in FIG. 1(f)-TRIS (1:1) admixture co-compressed (Carver press) (-○-). Dissolution testing was completed on a USP type-II apparatus at 37° C. with a paddle speed of 50 revolutions per minute (rpm). Quantitation of the drug concentration was completed using high pressure liquid chromatography (HPLC) analysis. A pH 4.5 citrate buffer was used to control the PH during the dissolution experiment. The volume of the buffer was 900 mL. Dissolution tests were completed with 10 mg (free acid equivalent) formulations. The salt (-▪-), despite it higher water solubility, did not dissolve as rapidly as the free acid capsules (-▴-). Dissolution of the co-compressed admixture (-○-) was extremely rapid as compared to the other formulations.

### DETAILED DESCRIPTION OF PREFERRED EMBODIMENTS

The oral bioavailabilities of poorly soluble non-ionizable drugs and the salts of poorly soluble, acidic or basic drugs have been found to be improved by preparing particles that include a mixture of the poorly soluble drug and an excipient. The particles, as discussed herein, are called “diffusion layer modulated solids.” The diffusion layer modulated solid particles contain a solid form of a drug or a drug salt closely associated with an acidic, basic, or solubilizing excipient. As used herein, “closely associated” means that the drug or drug salt and the excipient exist as separate components in the particles, but are closely associated on a micrometer scale within the particles. Dissolution of the particles results in a change in the pH and/or solubility of the drug within the aqueous diffusion layer that surrounds the particles during dissolution.

Upon contact of a drug crystal with water, a stagnant aqueous diffusion layer is formed surrounding the drug crystal and a saturated solution of the drug is generated at the immediate surface of the dissolving crystal. The dissolution rate of the drug is determined by the solubility of the drug in the immediate diffusion layer, the diffusion coefficient of the drug within the aqueous diffusion layer, and the total surface area presented by the drug crystal.

When a solubilizing excipient is co-compressed with a poorly soluble drug, the resulting solubility of the drug in the diffusion layer generated on contact with water can be increased by the solubilizing action of the excipient in the diffusion layer. The higher solubility of the drug in the diffusion layer can lead to faster dissolution rate and the formation of a supersaturated solution, which can precipitate quickly upon standing. The supersaturated state can be maintained for long periods of time by addition of polymers such hydroxypropyl methyl cellusose (HPMC), other cellulosic materials, polyvinylpryrrolidone (PVP), or polyethylene glycols. Thus, co-compression, roller compaction, or spray drying can bring a soluble salt of a poorly soluble drug in close contact with an acidic, basic or solubilizing excipient to form diffusion layer modulated solids, which may be lightly powdered. The resulting diffusion layer modulated solids can be formulated with HPMC, other polymers, other excipients, and lubricating agents. The resulting solid can be formulated in capsules, compressed into tablets, or prepared as powder formulations. The oral bioavialaiblity of these diffusion layer modulated (DLM) solids is preferably improved over the oral bioavailability of the drugs alone or the drugs in conventional tablet or capsule formulations, which are often incompletely absorbed.

The particles can be prepared by methods including co-compression (e.g., using a hand operated press or a roller compactor followed by granulation) and spray drying. In some cases it is possible to use wet granulation with limited amounts of water followed by drying to associate the drug crystals with the acidic, basic, or solubilizing excipient.

In one embodiment, a diffusion layer modulated solid includes a soluble salt of a poorly soluble, basic drug and an excipient selected from the group consisting of acidic excipients, solubilizing excipients, and combinations thereof.

In another embodiment, a diffusion layer modulated solid includes a soluble salt of a poorly soluble, acidic drug and an excipient selected from the group consisting of basic excipients, solubilizing excipients, and combinations thereof.

In another embodiment, a diffusion layer modulated solid includes a poorly soluble, non-ionizable drug and a solubilizing excipient.

In one embodiment, the diffusion layer modulated solid preferably includes a weight ratio of a poorly soluble drug or a soluble salt of a poorly soluble drug to excipient of at least 15:85, more preferably at least 25:75, and most preferably at least 35:65. In this embodiment, the diffusion layer modulated solid preferably includes a weight ratio of a poorly soluble drug or a soluble salt of a poorly soluble drug to excipient of at most 95:5, more preferably at most 90:10, and most preferably at most 85:15.

In another embodiment, the diffusion layer modulated solid preferably includes a weight ratio of a poorly soluble, non-ionizable drug:excipient of at least 15:85, more preferably at least 25:75, and most preferably at least 35:65. In this embodiment, the diffusion layer modulated solid preferably includes a weight ratio of a poorly soluble, non-ionizable drug:excipient of at most 95:5, more preferably at most 90:10, and most preferably at most 85:15.

Poorly soluble drugs are well known in the art and include, for example, those recited in U.S. Pat. Application Publication No. 2003/0091643 A1 (Friesen et al.) Preferred poorly soluble drugs include, for example, prochlorperazine edisylate, ferrous sulfate, albuterol, aminocaproic acid, mecamylamine hydrochloride, procainamide hydrochloride, amphetamine sulfate, methamphetamine hydrochloride, benzphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, diphenidol, meclizine hydrochloride, prochlorperazine maleate, phenoxybenzamine, thiethylperazine maleate, anisindione, diphenadione erythrityl tetranitrate, digoxin, isoflurophate, acetazolamide, nifedipine, methazolamide, bendroflumethiazide, chlorpropamide, glipizide, glyburide, gliclazide, tobutamide, chlorproamide, tolazamide, acetohexamide, metformin, troglitazone, orlistat, bupropion, nefazodone, tolazamide, chlormadinone acetate, phenaglycodol, allopurinol, aluminum aspirin, methotrexate, acetyl sulfisoxazole, hydrocortisone, hydrocorticosterone acetate, cortisone acetate, dexamethasone and its derivatives such as betamethasone, triamcinolone, methyltestosterone, 17-β-estradiol, ethinyl estradiol, ethinyl estradiol 3-methyl ether, prednisolone, 17-β-hydroxyprogesterone acetate, 19-nor-progesterone, norgestrel, norethindrone, norethisterone, norethiederone, progesterone, norgesterone, norethynodrel, terfandine, fexofenadine, aspirin, acetaminophen, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, selegiline, chlorpromazine, methyldopa, dihydroxyphenylalanine, calcium gluconate, ketoprofen, ibuprofen, cephalexin, erythromycin, haloperidol, zomepirac, vincamine, phenoxybenzamine, diltiazem, mirinone, captropril, mandol, quanbenz, hydrochlorothiazide, ranitidine, flurbiprofen, fenbufen, fluprofen, tolmetin, alclofenac, mefenamic, flufenamic, difuninal, nimodipine, nitrendipine, nisoldipine, nicardipine, felodipine, lidoflazine, tiapamil, gallopamil, amlodipine, mioflazine, lisinopril, enalapril, captopril, ramipril, enalaprilat, famotidine, nizatidine, sucralfate, etintidine, tetratolol, minoxidil, chlordiazepoxide, diazepam, amitriptyline, and imipramine, and pharmaceutical salts of these active agents, and combinations thereof.

Soluble Salts of Poorly Soluble Basic Drugs

Poorly soluble, basic drugs generally have a pKₐ of at least 1, preferably at least 2, and more preferably at least 3. Methods of measuring the pKₐ are well known to one of skill in the art and include, for example, conventional titration methods.

Poorly soluble, basic drugs generally have a solubility of at most 50 micrograms/ml, often times at most 25 micrograms/ml, and sometimes at most 10 micrograms/ml in an aqueous fluid at pH 6 to pH 7 at 25° C. Poorly soluble, basic drugs preferably have a solubility of at least 1 microgram/ml, more preferably at least 2 micrograms/ml, and most preferably at least 5 micrograms/ml in an aqueous fluid at pH 6 to pH 7 at 25° C. Methods for determining solubility are well known to one of skill in the art and include, for example, high pressure liquid chromatography (HPLC) after equilibration of an aqueous suspension of a drug or drug salt at, for example, 25° C. or 37° C., in water or buffered water, followed by filtration.

Examples of poorly soluble, basic drugs include, for example, those poorly soluble drugs listed herein above that have a pKₐ of at least 1, preferably at least 2, and more preferably at least 3. Preferred poorly soluble, basic drugs include, for example, acenocoumarol, albuterol, alprenolol, amitriptyline, amlodipine, amphetamine sulfate, atenolol, atropine sulfate, benzphetamine hydrochloride, bepridil, bupropion, chlorpromazine, cimetidine, clonidine, clotrimazole, diazepam, dihydroxyphenylalanine, diltiazem, econazole, erythromycin, felodipine, gallopamil, haloperidol, imipramine, imipramine, isoproterenol sulfate, isosorbide dinitrate, levodopa, lidoflazine, mecamylamine hydrochloride, meclizine hydrochloride, metformin, methamphetamine hydrochloride, methyldopa, miconazole, nefazodone hydrochloride, nicardipine, nisoldipine, phenformin hydrochloride, phenmetrazine hydrochloride, phenoxybenzamine, phenprocoumarol, pilocarpine hydrochloride, prazosin, procainamide hydrochloride, prochlorperazine edisylate, prochlorperazine maleate, propranolol, selegiline, terfandine, thiethylperazine maleate, tiapamil, timolol, tolterodine tartrate, and combinations thereof.

Soluble salts of poorly soluble, basic drugs may be prepared, for example, by allowing the basic drug to react with an organic or inorganic acid. Soluble salts of poorly soluble, basic drugs have a solubility of at least 1.5 times, more preferably at least 1.75 times, and most preferably at least 2 times that of the non-salt form of the drug in an aqueous fluid at pH 6 to pH 7 at 25° C.

Salts of poorly soluble, basic drugs typically include a counterion such as, for example, chloride, bromide, iodide, carbonate, sulfate, phosphate, nitrate, borate, thiocyanate, bisulfate, mesylate (i.e., methanesulfonate), camsylate (i.e., camphorsulfonate), isethionate (i.e., 2-hydroxyethanesulfonate), edisylate (i.e., 1,2-ethanedisulfonate), tosylate (i.e., p-toluenesulfonate), napsylate (2-naphthalenesulfonate), 1,5-naphthalenedisulfonate, esylate (i.e., ethanesulfonate), besylate (i.e., benzenesulfonate), estolate (i.e., lauryl sulfate), formate, acetate, propionate, malonate, succinate, adipate, maleate, fumarate, citrate, tartrate, lactate, gluconate, ascorbate, benzoate, hybenzate (i.e., o-(4-hydroxybenzoyl)benzoate), salicylate, lysinate, glycinate, glycerophosphate, aspartate, malate, orotate, saccharinate, cyclamate, gluceptate (i.e., D-glycero-D-gulo-heptanoate), glucuronate, mandalate, oxoglurate, camphorate, pantothenate, and combinations thereof.

Soluble Salts of Poorly Soluble Acidic Drugs

Acidic drugs generally have a pKₐ of at most 11, preferably at most 9, and more preferably at most 7. Methods of measuring the pKₐ are well known to one of skill in the art and include, for example, conventional titration methods.

Poorly soluble, acidic drugs generally have a solubility of at most 50 micrograms/ml, often times at most 25 micrograms/ml, and sometimes at most 10 micrograms/ml in an aqueous fluid at pH 6 to pH 7 at 25° C. Poorly soluble, acidic drugs preferably have a solubility of at least 1 microgram/ml, more preferably at least 2 micrograms/ml, and most preferably at least 5 micrograms/ml in an aqueous fluid at pH 6 to pH 7 at 25° C. Methods for determining solubility are well known to one of skill in the art and include, for example, high pressure liquid chromatography (HPLC) after equilibration of an aqueous suspension of a drug or drug salt at, for example, 25° C. or 37° C., in water or buffered water, followed by filtration.

Examples of poorly soluble, acidic drugs include, for example, those poorly soluble drugs listed herein above, that have a pKₐ of at most 11, preferably at most 9, and more preferably at most 7. Preferred poorly soluble, acidic drugs include, for example, acetazolamide, acetohexamide, alclofenac, aminocaproic acid, aspirin, benzapril, chlorpropamide, coumarin, ethyl biscoumacetate, fenbufen, fenoprofen, flufenamic acid, fluprofen, flurbiprofen, furosemide, gliclazide, glipizide, glyburide, hydrochlorothiazide, indomethacin, indoprofen, ketoprofen, lisinopril, lostartan k, mefenamic, methyltestosterone, minoxidil, mioflazine, mirinone, naproxen, phenobarbital, phenylbutazone, ramipril, sulindac, tolazamide, tolmetin, zomepirac, and combinations thereof.

Soluble salts of poorly soluble, acidic drugs may be prepared, for example, by allowing the acidic drug to react with an organic or inorganic base. Soluble salts of poorly soluble, acidic drugs have a solubility of at least 1.5 times, more preferably at least 1.75 times, and most preferably at least 2 times that of the non-salt form of the drug in an aqueous fluid at pH 6 to pH 7 at 25° C.

Salts of poorly soluble, basic drugs typically include a counterion such as, for example, lithium, sodium, potassium, bismuth, calcium, magnesium, zinc, aluminum, ammonium, choline, betaine (i.e., (carboxymethyl) trimethylammonium hydroxide), and combinations thereof.

A salt of the poorly soluble, basic drug may be formed, for example, from sodium hydrogen phosphate, erbumine (i.e., t-butylamine), diethylamine, piperazine, imidazole, ethylenediamine, pyridoxine, 4-phenylcyclohexylamine, olamine (i.e., 2-aminoethanol), diethanolamine, triethanolamine, tromethamine (i.e., tris(hydroxymethyl) aminomethane), meglumine (i.e., N-methylglucamine), eglumine (i.e., N-ethylglucamine), benzathine (i.e., N,N′-dibenzylethylenediamine),procaine, hydroxyethylpyrrolidone, hydrabamine (i.e., N,N′-di(dihydroabietyl)ethylenediamine, heptaminol (i.e., 6-amino-2-methylheptan-2-ol), chlorcyclizine (i.e., 1-(4-chorobenzyhydryl)-4-methylpiperazine), benethamine (i.e., N-benzylphenethylamine), and combinations thereof.

Poorly Soluble Non-Ionizable Drugs

Non-ionizable drugs are drugs that lack groups that are readily ionizable in an aqueous medium. Ionizable groups include, for example, those that are readily protonated (e.g., basic amine groups) and those that are readily deprotonated (e.g., carboxylic acid groups). Poorly soluble, non-ionizable drugs generally have a solubility of at most 50 micrograms/ml, often times at most 25 micrograms/ml, and sometimes at most 10 micrograms/ml in an aqueous fluid at pH 6 to pH 7 at 25° C. Poorly soluble, non-ionizable drugs preferably have a solubility of at least 1 microgram/ml, more preferably at least 2 micrograms/ml, and most preferably at least 5 micrograms/ml in an aqueous fluid at pH 6 to pH 7 at 25° C. Methods for determining solubility are well known to one of skill in the art and include, for example, high pressure liquid chromatography (HPLC) after equilibration of an aqueous suspension of a drug or drug salt at, for example, 25° C. or 37° C., in water or buffered water, followed by filtration.

Examples of poorly soluble, non-ionizable drugs include, for example, those poorly soluble drugs listed herein above, that lack groups that are readily ionizable in an aqueous medium. Preferred poorly soluble, non-ionizable drugs include, for example, 17-β-hydroxyprogesterone acetate, 17-β-estradiol, 19-nor-progesterone, acetaminophen, acetyl sulfisoxazole, allopurinol, anisindione, bendroflumethiazide, chlorindione, chlormadinone acetate, clopidogrel, cortisone acetate, dexamethasone, digoxin, ethinyl estradiol, ethinyl estradiol 3-methyl ether, hydrocorticosterone acetate, hydrocortisone, ibuprofen, nilvadipine, norethiederone, norethindrone, norethisterone, norethynodrel, norgesterone, norgestrel, prednisolone, progesterone, tobutamide, triamcinolone, troglitazone, and combinations thereof.

Excipients

Excipients may be included in compositions that include a diffusion layer modulated solid for a variety of reasons including, for example, to improve the flow properties of the formulation by including glidants; to improve the stability of the drug by including antioxidants; to change the color of the formulation by including dyes; to improve the taste perception of the tablet or capsule formulation by including taste enhancing agents; to improve the dissolution of the formulation by including surfactants. Excipients useful in the present invention are generally pharmaceutically acceptable excipients and are well known to one of skill in the art and include, for example, those listed in European Patent Application No. EP 1027886A2 (Babcock et al.); “Handbook of Pharmaceutical Additives,” M. Ash and I. Ash, Gower Publications, Vermont (1997); and “Handbook of Pharmaceutical Excipients,” 3^{rd} Edition, A. H. Kirbe, Am.Pharm.Assoc., Washington D.C. (2000).

Compositions including diffusion layer modulated solids may optionally include excipients to aid in maintaining the supersaturatated state. Examples of such useful excipients include, for example, poly(vinyl pyrrolidone), carboxymethyl cellulose, cellulose acetate phthalate, carboxyethyl cellulose, hydroxyethyl ethyl cellulose, hydroxyethyl cellulose, hydroxy ethyl cellulose acetate, hydroxypropylcellulose, hydroxypropylmethyl cellulose, methyl cellulose, chitosan, hydroxy ethyl methyl cellulose, hydroxypropyl methyl cellulose phthalate, ethylene vinyl alcohol copolymer, vinyl alcohol-vinyl acetate copolymer, cellulose acetate trimellitate, cellulose acetate terephthalate, hydroxypropyl methyl cellulose acetate, hydroxypropyl methyl cellulose acetate phthalate, hydroxypropyl methyl cellulose acetate succinate, cellulose propionate phthalate, hydroxypropyl methyl cellulose succinate, cellulose propionate trimellitate, cellulose butyrate trimellitate, hydroxypropyl cellulose acetate phthalate, methyl cellulose acetate phthalate, hydroxyethyl methyl cellulose acetate succinate, hydroxypropyl cellulose butyrate phthalate, cellulose acetate isophthalate, ethyl cellulose acetate phthalate, hydroxypropyl cellulose acetate phthalate succinate, methyl cellulose acetate trimellitate, ethyl cellulose acetate trimellitate, hydroxypropyl cellulose acetate trimellitate, hydroxypropyl cellulose acetate trimellitate succinate, cellulose acetate pyridinedicarboxylate, ethyl cellulose acetate benzoate, ethyl hydroxypropyl ethyl cellulose acetate benzoate, ethyl cellulose acetate nicotinate, ethyl cellulose acetate picolinate, gum arabic, carrageenan, gum ghatti, guar gum, gum karaya, gum tragacanth, block ethylene oxide/propylene oxide co-polymers (e.g., those available under the trade designation PLURONIC F68, PLURONIC F108, PLURONIC F127, and PLURONIC F50 from BASF Corp., Mount Olive, N.J.), polyethylene glycols such as polyethylene glycol 400, 600, 800, 1000, 4000 and the like and the corresponding monoalkyl polyethylene glycols such as cetomacrogol or polyethylene glycol 1000 cetyl ether, and combinations thereof.

Compositions including diffusion layer modulated solids may optionally include pharmaceutically acceptable diluents as excipients. Suitable diluents include, for example, lactose USP; lactose USP, anhydrous; lactose USP, spray dried; starch USP; directly compressible starch; mannitol USP; sorbitol; dextrose monohydrate; microcrystalline cellulose NF; dibasic calcium phosphate dihydrate NF; sucrose-based diluents; confectioner's sugar; and combinations thereof. Such diluents, if present, preferably constitute at least 5%, more preferably at least 10%, and most preferably at least 20%, of the total weight of the composition. Such diluents, if present, preferably constitute at most 99%, more preferably at most 85%, and most preferably at most 80%, of the total weight of the composition. The diluent or diluents selected preferably exhibit suitable flow properties and, where tablets are desired, compressibility. Preferred diluents include lactose, microcrystalline cellulose, and combinations thereof.

Compositions including diffusion layer modulated solids may optionally include excipients to improve hardness (e.g., for tablets) and to provide suitable release rates, stability, pre compression flowability, drying properties, and/or disintegration time. Such useful excipients include, for example, extragranular microcrystalline cellulose (e.g., microcrystalline cellulose added to a wet granulated composition after the drying step) lactose (e.g., lactose monohydrate), and combinations thereof.

Compositions including diffusion layer modulated solids may optionally include pharmaceutically acceptable disintegrants as excipients, particularly for tablet formulations. Suitable disintegrants include, for example, starches; sodium starch glycolate; clays (such as Veegum HV); celluloses (such as purified cellulose, methylcellulose, sodium carboxymethylcellulose and carboxymethylcellulose); alginates; pregelatinized corn starches (such as National 1551 and National 1550); crospovidone USP NF; and gums (such as agar, guar, locust bean, Karaya, pectin, and tragacanth); and combinations thereof. Disintegrants may be added at any suitable step during the preparation of the compositions, particularly prior to granulation or during the lubrication step prior to compression. Such disintegrants, if present, preferably constitute in total at least 0.2% of the total weight of the composition. Such disintegrants, if present, preferably constitute in total at most 30%, more preferably at most 10%, and most preferably at most 5%, of the total weight of the composition. A preferred disintegrant for tablet or capsule disintegration is croscarmellose sodium. If present, croscarmellose sodium preferably constitutes at least 0.2% of the total weight of the composition. If present, croscarmellose sodium preferably constitutes at most 10%, more preferably at most 6%, and most preferably at most 5%, of the total weight of the composition. Croscarmellose sodium preferably confers superior intragranular disintegration capabilities to compositions of the present invention.

Compositions including diffusion layer modulated solids may optionally include pharmaceutically acceptable binding agents or adhesives as excipients (e.g., for tablet formulations). Such binding agents and adhesives preferably impart sufficient cohesion to the powder being tableted to allow for normal processing operations such as sizing, lubrication, compression, and packaging, but still allow the tablet to disintegrate and the composition to be absorbed upon ingestion. Suitable binding agents and adhesives include, for example, acacia; tragacanth; sucrose; gelatin; glucose; starch; cellulose materials such as, but not limited to, methylcellulose and sodium carboxymethylcellulose (e.g., Tylose); alginic acid and salts of alginic acid; magnesium aluminum silicate; polyethylene glycol; guar gum; polysaccharide acids; bentonites; polyvinylpyrrolidone; polymethacrylates; hydroxypropylmethylcellulose (HPMC); hydroxypropylcellulose (Klucel); ethylcellulose (Ethocel); pregelatinized starch (such as National 1511 and Starch 1500), and combinations thereof. Such binding agents and/or adhesives, if present, preferably constitute in total at least 0.5%, more preferably at least 0.75%, and most preferably at least 1%, of the total weight of the composition. Such binding agents and/or adhesives, if present, preferably constitute in total at most 25%, more preferably at most 15%, and most preferably at most 10%, of the total weight of the composition. A preferred binding agent is polyvinylpyrrolidone, the use of which may impart cohesive properties to a powder blend and may facilitate binding to form granules during, for example, wet granulation. Polyvinylpyrrolidone, if present, preferably constitutes at least 0.5% of the total weight of the composition. Polyvinylpyrrolidone, if present, preferably constitutes at most 10%, more preferably at most 7%, and most preferably at most 5%, of the total weight of the composition. Polyvinylpyrrolidones having viscosities up to 20 centipoise (cPs) are preferred, those having viscosities of 6 cPs or lower are particularly preferred, even more particularly preferred are those having viscosities of 3 cPs or lower.

Compositions including diffusion layer modulated solids may optionally include pharmaceutically acceptable wetting agents as excipients. Such wetting agents are preferably selected to maintain the diffusion layer modulated solid in close association with water, a condition that is believed to improve the relative bioavailability of the composition. Suitable wetting agents include, for example, oleic acid; glyceryl monostearate; sorbitan monooleate; sorbitan monolaurate; triethanolamine oleate; polyoxyethylene sorbitan monooleate; polyoxyethylene sorbitan monolaurate; sodium oleate; sodium lauryl sulfate (SLS) or sodium dodecyl sulfate (SDS) (used interchangeably herein); and combinations thereof. Wetting agents that are anionic surfactants are preferred. Wetting agents, if present, preferably constitute in total at least 0.25%, more preferably at least 0.4%, and most preferably at least 0.5%, of the total weight of the composition. Wetting agents, if present, preferably constitute in total at most 15%, more preferably at most 10%, and most preferably at most 5%, of the total weight of the composition. A preferred wetting agent is sodium lauryl sulfate. Sodium lauryl sulfate, if present, preferably constitutes at least 0.25%, more preferably at least 0.4%, and most preferably at least 0.5%, of the total weight of the composition. Sodium lauryl sulfate, if present, preferably constitutes at most 7%, more preferably at most 6%, and most preferably at most 5%, of the total weight of the composition.

Compositions including diffusion layer modulated solids may optionally include pharmaceutically acceptable lubricants and/or glidants as excipients. Suitable lubricants and/or glidants include, either individually or in combination, glyceryl behapate (Compritol 888); stearates (magnesium, calcium, and sodium); stearic acid; hydrogenated vegetable oils (e.g., Sterotex); talc; waxes; Stearowet; boric acid; sodium benzoate; sodium acetate; sodium fumarate; sodium chloride; leucine; polyethylene glycols (e.g., Carbowax 4000 and Carbowax 6000); sodium oleate; sodium lauryl sulfate; and magnesium lauryl sulfate. Such lubricants, if present, preferably constitute in total at least 0.1%, more preferably at least 0.2%, and most preferably at least 0.25%, of the total weight of the composition. Such lubricants, if present, preferably constitute in total at most 10%, more preferably at most 8%, and most preferably at most 5%, of the total weight of the composition. A preferred lubricant is magnesium stearate, which may be used, for example, to reduce friction between the equipment and granulated mixture during compression of tablet formulations.

Compositions including diffusion layer modulated solids may optionally include other excipients (such as anti-adherent agents, colorants, flavors, sweeteners and preservatives) that are known in the pharmaceutical art.

ACIDIC EXCIPIENTS. Acidic excipients have a pKₐ of at most 6, preferably at most 5.5, and more preferably at most 5. Methods of measuring the pKₐ are well known to one of skill in the art and include, for example, conventional titration methods. Acidic excipients useful in the present invention include, for example, those excipients listed herein above that have a pKa of at most 6, preferably at most 5.5, and more preferably at most 5.

Examples of suitable acidic excipients include maleic acid, citric acid, tartaric acid, pamoic acid, fumaric acid, tannic acid, salicylic acid, 2,6-diaminohexanoic acid, camphorsulfonic acid, gluconic acid, glycerophosphoric acid, 2-hydroxyethanesulfonic acid isethionic acid, succinic acid, carbonic acid, p-toluenesulfonic acid, aspartic acid, 8-chlorotheophylline, benzenesulfonic acid, malic acid, orotic acid, oxalic acid, benzoic acid, 2-naphthalenesulfonic acid, stearic acid, adipic acid, p-aminosalicylic acid, 5-aminosalicylic acid, ascorbic acid, sulfuric acid, cyclamic acid, sodium lauryl sulfate, glucoheptonic acid, glucuronic acid, glycine, sulfuric acid, mandelic acid, 1,5-naphthalenedisulfonic acid, nicotinic acid, oleic acid, 2-oxoglutaric acid, pyridoxal 5-phosphate, undecanoic acid, p-acetamidobenzoic acid, o-acetamidobenzoic acid, m-acetamidobenzoic acid, N-acetyl-L-aspartic acid, camphoric acid, dehydrocholic acid, malonic acid, edetic acid, ethylenediaminetetraacetic acid, ethylsulfuric acid, hydroxyphenylbenzoylbenzoic acid, glutamic acid, glycyrrhizic acid, 4-hexylresorcinol, hippuric acid, p-phenolsulfonic acid, 4-hydroxybenzoic acid, 3-hydroxybenzoic acid, 3-hydroxy-2-naphthoic acid, 1-hydroxy-2-naphthoic acid, lactobionic acid, 3′-adenylic acid, 5′-adenylic acid, mucic acid, galactaric acid, pantothenic acid, pectic acid, polygalacturonic acid, 5-sulfosalicylic acid, 1,2,3,6-tetrahydro-1,3-dimethyl-2,6-dioxopurine-7-propanesulfonic acid, terephthalic acid, 1-hydroxy-2-naphthoic acid, and combinations thereof.

Preferred acidic excipients include, for example, maleic acid, citric acid, malic acid, fumaric acid, saccharin, sulfuric acid including bisulfate salts, tartaric acid, lactic acid, salicylic acid, lysine, d-camphorsulfonic acid, aspartic acid, aminosalicylic acid, cyclamic acid, glycine, mandelic acid, malonic acid, glutamic acid, glucose-1-phosphate, and combinations thereof.

BASIC EXCIPIENTS. Basic excipients have a pKₐ of at least 4, preferably at least 5, and more preferably at least 6. Methods of measuring the pKₐ are well known to one of skill in the art and include, for example, conventional titration methods. Basic excipients useful in the present invention include, for example, those excipients listed herein above that have a pKₐ of at least 4, preferably at least 5, and more preferably at least 6.

Examples of suitable basic excipients include N-methylglucamine, ammonia, tris(hydroxymethyl)aminomethane, piperazine, diethylamine, choline chloride, 4-phenylcyclohexylamine, ethanolamine, diethanolamine, N,N′-dibenzylethylenediamine, imidazole, triethanolamine, potassium citrate, sodium citrate, pyridoxine hydrochloride, procaine, 6-amino-2-methyl-2-heptanol, 1,2-ethanediamine, tert-butylamine, N-ethylglucamine, diethylamine, dibenzylamine, 1-[(4-chlorophenyl)phenylmethyl]-4-methylpiperazine, N-benzyl-2-phenethylamine, and combinations thereof.

Preferred basic excipients include, for example, tris(hydroxymethyl)aminomethane (tris), trisodiumphosphate, N-methyl glucamine, piperazine, imidazole, procaine, ornithine, arginine, glucosamine, and combinations thereof.

SOLUBLILIZING EXCIPIENTS. Solubilizing excipients are excipients that result in increased drug solubility for a mixture of the drug and the excipient compared to the drug in the absence of the excipient. Suitable solubilizing excipients include, for example, those listed herein above and in “Handbook of Pharmaceutical Additives,” M. Ash and I. Ash, Gower Publications, Vermont (1997). Preferably, solubilizing excipients are non-polymeric.

In addition to the preferred acidic and basic excipients listed herein above, preferred solubilizing excipients include, for example, urea, acetylurea, sorbic acid, sodium sorbate, sodium succinate, sodium benzoate, benzoic acid, sodium lauryl sulfate, sodium stearyl fumarate, sodium stearyl lactylate, sodium lauroyl sarcosinate, sodium lauryl sulfate, sodium cocomonoglyceride sulfonate, sodium cocoate, sodium caprate, sodium bisulfate (sodium hydrogensulfate), sodium laurylsulfoacetate, sodium dioctylsulfosuccinate, THAM, disodium hydrogen phosphate, trisodium phosphate, sucrose oleate, trisodium citrate, citric acid, lauroylsarcosine, malic acid (hydroxysuccinic acid, apple acid), fumaric acid, crotonic acid, 2-amino-2-methyl-1,3-propanediol, L-aspartic acid, L-lysine, L-glutamic acid, dimethylbenzamide, nicotinamide, ethylurea, and combinations thereof. In some embodiments, solubilizing excipients may be polymeric. Suitable polymeric solubilizing excipients include, for example, polyethylene glycol 1000, polyethylene glycol 3350, polyethylene glycol 6000, polyethylene glycol 10000, and combinations thereof.

Crystal Growth Inhibitors

Diffusion layer modulated solids may optionally include or be formulated with crystal growth inhibitors to prevent or retard crystallization of the drug, preferably resulting in increased bioavailability. The crystal growth inhibitor can be added, for example, before and/or after co-compression or spray drying of the drug and excipient. For example, a diffusion layer modulated solid can be blended with a crystal growth inhibitor, with the resulting mixture being placed in capsules or compressed into tablets.

Crystal growth inhibitors are well known to one of skill in the art and include, for example, cellulosic polymers. Crystal growth inhibitors useful in the present invention include, for example, hydroxypropyl methyl cellulose (HPMC), hydroxypropyl methyl cellulose acetate succinate (HPMCAS), cellulose acetate trimellitate (CAT), cellulose acetate phthalate (CAP), hydroxypropyl cellulose acetate phthalate (HPCAP), hydroxypropyl methyl cellulose acetate phthalate (HPMCAP), methyl cellulose acetate phthalate (MCAP); carboxymethyl ethyl cellulose (CMEC); methyl cellulose acetate phthalate (MCAP), polyvinlypyrrolidone (PVP), polyethylene glycol (PEG), and combinations thereof.

Methods

A diffusion layer modulated solid of the present invention may be prepared from a poorly soluble drug or a soluble salt of a poorly soluble drug; and an excipient by a variety of methods including, for example, co-compression and spay drying. Preferably the soluble salt of the poorly soluble drug and/or the excipient are in the form of paticles before being admixed. Preferably the average size of the particles is at most 400 micrometers, more preferably at most 100 micrometers, even more preferably at most 50 micrometers, and most preferably at most 20 micrometers. Preferably the average size of the particles is at least 0.1 micrometers, more preferably at least 1 micrometer, even more preferably at least 5 micrometers, and most preferably at least 10 micrometers. When co-compression of a drug and an excipient is used to prepare a diffusion layer modulated solid, preferably the co-compression uses a pressure of at least 70 megapascals (MPa) (10,000 pounds per square inch (psi)), more preferably at least 140 MPa (20,000 psi), even more preferably at least 210 MPa (30,000 psi), and most preferably at least 240 MPa (35,000 psi).

In one embodiment of the present invention, co-compression of the diffusion layer modulated solid may be provided by a technique including roller compaction, followed by granulation. Roller compaction is a technique that is widely used in the pharmaceutical industry for granulation. See, for example, Miller et al., “A Survey of Current Industrial Practices and Preferences of Roller Compaction Technology and Excipients Year 2000,” American Pharmaceutical Review, pp. 24-35, Spring 2001. By using, for example, a roller compactor, to co-compress a poorly soluble drug or a soluble salt of a poorly soluble drug with an excipient under high pressure, it is possible to provide an intimate mixture of the two materials in the form of a “glassy” ribbon. Lightly powdering the resulting “ribbon” may result in a coarse granulation of the co-compressed diffusion layer modulated powder. Micronized materials (e.g., drugs, drug salts, and/or excipients) are preferred, and submicron forms of the materials are potentially useful.

Preferably the roller compaction process provides co-compression using at least 9000 newtons (2000 pounds force), more preferably at least 18000 newtons (4000 pounds force), and most preferably at least 27000 newtons (6000 pounds force). See, for example, Gereg et al., Pharmaceutical Technology, (Oct. 1, 2002); and Adeyeye, American Pharmaceutical Review, 3:37-39, 41-42 (2000). Dissolution of drugs with roller compaction has also been reported by Mitchell et al., International Journal of Pharmaceutics, 250:3-11 (2003).

In another embodiment of the present invention, a diffusion layer modulated solid may be provided by a technique including spray drying. Spray drying is a technique that is widely used in the pharmaceutical industry to provide powdered, granulated, and agglomerated products including, for example, drugs. See, for example, PCT International Publication No. WO0142221 (Hageman et al.); and Nath et al., Drying Technology, 16:1173-1193 (1998). In general a mixture of two materials may be provided in a fluid (e.g., a volatile liquid) as a solution, emulsion, or suspension. Preferably the fluid is a volatile liquid that includes water. The fluid is preferably pressurized though an atomizer to form a spray having the required droplet size distribution. Evaporation, which is preferably controlled by airflow and temperature, results in formation of the desired particles.

Characterization of Diffusion Layer Modulated Solids

For some embodiments of the present invention, a diffusion layer modulated solid is in the form of particles. Preferably, the particles have an average size of at least 5 micrometers, more preferably at least 20 micrometers, and most preferably at least 50 micrometers. Preferably, the particles have an average size of at most 400 micrometers, more preferably at most 300 micrometers, and most preferably at most 200 micrometers. Optionally, the particles may form granules.

For some embodiments of the present invention, particles of a diffusion layer modulated solid are preferably homogeneous at a spatial domain of at most 50 micrometers, more preferably at most 30 micrometers, and most preferably at most 20 micrometers.

Dissolution rates of diffusion layer modulated solids may be measured by a variety of techniques that are well known to one of skill in the art. See, for example, Bryn et al., “Solid-State Chemistry of Drugs,” pp. 91-102, SSCI Inc., West Lafayette, Ind. (1999). Dissolution rates may be determined, for example, by a USP dissolution type II (paddle) apparatus or a rotating disk method. Preferably dissolution rates are measured at 25° C. in water at a pH of 1 to 7. Preferably, the pH is selected to be the pH at which the solubility of the free drug is at a minimum.

For some embodiments, the rotating disk method is preferably used to determine dissolution rates. Specifically, the rotating disk method is used to evaluate dissolution in the following manner. Mixtures of the powdered material are prepared and then compressed in a 0.48 cm (3/16 inch) diameter punch and die with a Carver press for 1 minute at 4450 newtons (1000 pounds force) (i.e., 255 MPa (37000 psi)). Dissolution is measured by rotating the disk at 300 rpm with an electric motor and putting it into 50 ml of dissolution fluid. The pH of the media can be varied from 0-8 depending on the contents of the dissolution media. The concentration of drug as a function of time is determined by measuring the UV absorbance spectroscopy of the compound of interest as a function of time. The intrinsic dissolution rate is calculated by dividing the slope of the concentration vs. time line by the surface area of the compound of interest exposed in the solution. For at least one pH using this preferred method, a diffusion layer modulated solid including a poorly soluble drug or a soluble salt of a poorly soluble drug preferably has an intrinsic dissolution rate at least 10% greater, more preferably at least 50% greater, and most preferably at least 100% greater than the intrinsic dissolution rate of the poorly soluble drug or the soluble salt of the poorly soluble drug alone at the same pH, and wherein the dissolution rates are both measured at 25° C. in water at a pH of 1 to 7. Preferably, the pH is selected to be the pH at which the solubility of the free drug is at a minimum.

Diffusion layer modulated solids of the present invention may be used in a variety of forms including, for example, capsules, tablets, and powder or sachet or granule formulations. Capsules may be prepared that include diffusion layer modulated solids of the present invention. Tablets that include diffusion layer modulated solids of the present invention may also be prepared by techniques well known to one of skill in the art as described, for example, on the world wide web at pformulate.com.

Bioavailability of diffusion layer modulated solids may be determined by a variety of techniques that are well known to one of skill in the art. Preferably the bioavailability of the diffusion layer modulated solids of the present invention is increased in comparison to the bioavailability of the poorly soluble drug or soluble salt of the poorly soluble drug alone. More preferably the bioavailability of the diffusion layer modulated solids of the present invention is at least 50% greater, and most preferably at least 100% greater in comparison to the bioavailability of the poorly soluble drug or soluble salt of the poorly soluble drug alone. Diffusion layer modulated solids may preferably be used to provide improved methods of treating or preventing disease in animals, and preferably in humans.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Improved Dissolution of a Soluble Salt of a Poorly Soluble, Basic Drug by Using a Co-compressed Mixture of the Drug Salt and an Acidic Excipient

Materials and Methods

Delavirdine mesylate is a soluble salt of the poorly soluble, basic drug delavirdine, which can be prepared as described, for example, in PCT International Publication No. WO91/09849 (Romero et al.). Tablets including delavirdine mesylate (e.g., 100 mg or 200 mg) are available under the trade designation RESCRIPTOR from Pfizer Inc., New York, N.Y. Citric acid monohydrate is an acidic excipient that is available from Mallinckrodt, Hazelwood, Mo.

Intrinsic Dissolution Rate Determination of Delavirdine Mesylate

The intrinsic dissolution rates of delavirdine mesylate and the delavirdine mesylate-citric acid co-compressed admixtures were determined by a fiber optic automated rotating disk dissolution method.

Preparation of Delavirdine Mesylate Compressed Disks for Intrinsic Dissolution Rate Determination

The delavirdine mesylate and the delavirdine mesylate-citric acid (2:1) admixtures were co-compressed in a stainless steel (SS) die, 3.2 cm (1¼ inch) diameter×2.5 cm (1 inch), containing a central 0.48 cm (3/16 inch) hole using a punch consisting of a 0.48 cm (3/16 inch) high speed steel (HSS) rod (8.9 cm; 3½ inches long). The 0.48 cm (3/16 inch) HSS rod was inserted into the die to a distance of 1.9 cm (¾ inch), leaving 0.64 (¼ inch) for placement of 20±1 mg of the drug or drug mixture into the 0.48 cm (3/16 inch) diameter hole.

After adding the drug, the punch (or HSS rod) was inserted into the die and the entire die assembly was placed into a 3-bolt holder that was used to hold a 0.64 cm (¼ inch) SS base plate firmly against the powder bed during compression in the die. Compression of the powder was achieved on a Carver press using a stepwise increase in the force up to 4450 newtons (1000 pounds force) (i.e., 255 MPa (37000 psi)) and then a progressive decrease in pressure as described in the following. A force of 1110 newtons (250 pounds force) was applied for approximately 10 seconds and the pressure was removed. This was repeated at 2220 newtons (500 pounds force), 3330 newtons (750 pounds force), and 4450 newtons (1,000 pounds force). The 4450 newtons (1000 pounds force) (i.e., 255 MPa (37000 psi)) was applied again and maintained for 1 minute. The pressure was decreased stepwise by simply lowering the pressure and then holding it at 3330 newtons (750 pounds force) for 10 seconds and repeating this at 2220 newtons (500 pounds force), 1110 newtons (250 pounds force) and, finally, the pressure was removed.

The die and holder was removed from the Carver press and the punch (or HSS rod) was twisted to loosen the rod and to allow the pellet to relax or expand from the backside. After a three minute (minimum) relaxation period, the set-screw on the HSS rod was firmly secured to the die.

The entire punch and die assembly containing the drug pellet with one face of the drug pellet exposed was removed as a unit from the holder and the intrinsic dissolution rate was determined as described below.

Determination of the Intrinsic Dissolution Rate of Delavirdine Mesylate

The HSS rod in the die containing the drug compact with one face of the drug pellet exposed was attached to an electric motor with a fixed speed of 300 revolutions per minute (rpm). The die was rotated (300 rpm) while the die (containing the drug pellet) was lowered at t=0 into the center of the dissolution vessel consisting of a jacketed 800 mL beaker (Pyrex, No.1000) containing 500 mL of the desired de-gassed (house vacuum, 3 minutes) dissolution medium maintained at 37±0.5° C. The dissolution medium consisted of either dilute HCl (0.01, 0.001 or 0.0001 N HCl) or pH 6, 0.01 M phosphate containing 0.6% SLS (sodium lauryl sulfate). The die was positioned such that the drug compact was approximately 6.4 cm (2.5 inches) from the bottom of the 500 mL dissolution beaker and approximately the same distance from the liquid surface. Continuous monitoring by ultraviolet (UV) spectroscopy was conducted by the fiber optic UV automated dissolution method or samples were taken automatically by the HPLC sampling method as described below.

The Fiber Optic Dissolution System. The fiber optic UV automated dissolution system employed an Ocean Optics PC Model 1000 fiber optic spectrophotometer connected to a 120 mHz Pentium computer. The dissolution process was monitored continuously at 290 nm with the fiber optic probe with 5-10 data points taken per minute. The data was processed automatically with a Visual Basic application program that allowed the data to be collected automatically from the spectrophotometer.

The delavirdine mesylate intrinsic dissolution rate profile was plotted in Excel and the intrinsic dissolution rate was calculated automatically by the program. The dissolution period was usually 15 minutes, but could be as short as approximately 1 minute, or as long as a few hours.

Calculation of the Intrinsic Dissolution Rate. The intrinsic dissolution rates (IDR) were calculated from the slope of the plot of the concentration in solution vs. time, the volume (500 mL), and the surface area of the drug disk (0.177 cm²) using the following equation:
IDR=(Slope·500 mL)/(0.177 cm²·60 seconds·minute⁻¹)
with slope in units of (microgram·mL⁻¹·minute⁻¹) and IDR in units of (micrograms·cm⁻²·seconds⁻¹).
Light Microscopy of Delavirdine Mesylate and Citric Acid Mixtures

Light microscopy was conducted on an Olympus BHSP polarized light microscope. Powder was spread in a thin layer on a glass microscope slide. A coverslip was then loaded with approximately 5 microliters of solution and carefully lowered onto the powder. Observations were made using a video camera. Images were retained by digitized images from the video camera feed.

Results

Predicted Intrinsic Dissolution Rate of Delavirdine Mesylate

The theory for the calculation of the dissolution rate of a salt was based on the Mooney model (Mooney et al., J. Pharm. Sci., 70:13-22 (1981); Mooney et al., J. Pharm. Sci., 70:22-32 (1981)). Interestingly, the intrinsic dissolution rate of delavirdine mesylate was predicted to be very fast (approximately 400 micrograms○sec⁻¹○cm−2) and nearly pH independent. The rapid dissolution of delavirdine mesylate at pH 6 was not observed in practice due to formation of a film of the delavirdine free base over the surface of the mesylate salt as described hereinafter. The results reported herein for the co-compression of delavirdine mesylate with citric acid are consistent with the prevention of surface precipitation of the delavirdine free base.

Intrinsic Dissolution Rate Studies

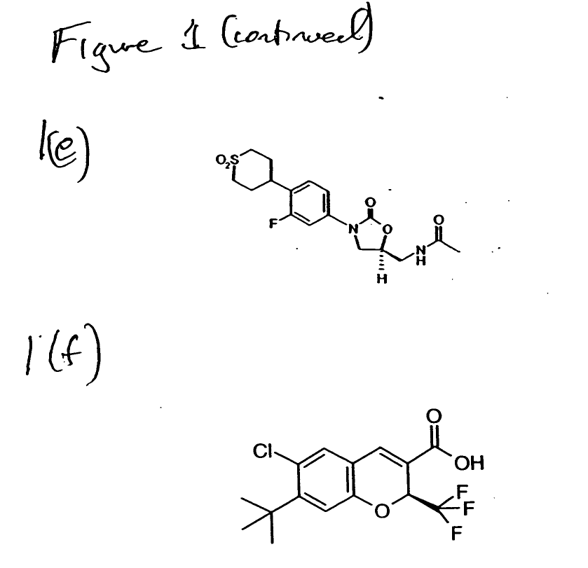

FIG. 2 shows the intrinsic dissolution profiles of the delavirdine mesylate-citric acid admixture (2:1 w/w ratio) at pH 6 (0.01 M phosphate) containing 0.6% SLS (sodium lauryl sulfate) along with the intrinsic dissolution rate of delavirdine mesylate alone at pH 2 (0.01 N) HCl and at pH 6 (0.01 M phosphate) containing 0.6% SLS.

At pH 2, pure delavirdine mesylate rapidly dissolves initially but the dissolution stops after approximately 60% of the drug is dissolved due to formation of delavirdine free base on the surface of the pellet.

At pH 6 (0.01 M phosphate, 0.6% SLS), the intrinsic dissolution rate of pure delavirdine mesylate is exceptionally slow with much less than 1% of the 20 mg drug pellet dissolved in 60 minutes due to surface precipitation of the delavirdine free base.

The dissolution of the delavirdine mesylate-citric acid co-compressed admixture, however, is fast at pH 6 (with 0.6% SLS). Complete dissolution (100%) of the 20 mg pellet containing approximately 12 mg of delavirdine mesylate occurred in less than 10 minutes, whereas less than 1% of the pure delavirdine mesylate pellet dissolved in the same time period. In conclusion, the dissolution rate of the delavirdine mesylate-citric acid (2:1) co-compressed admixture at pH 6 with 0.6% SLS is at much faster than that of delavirdine mesylate alone.

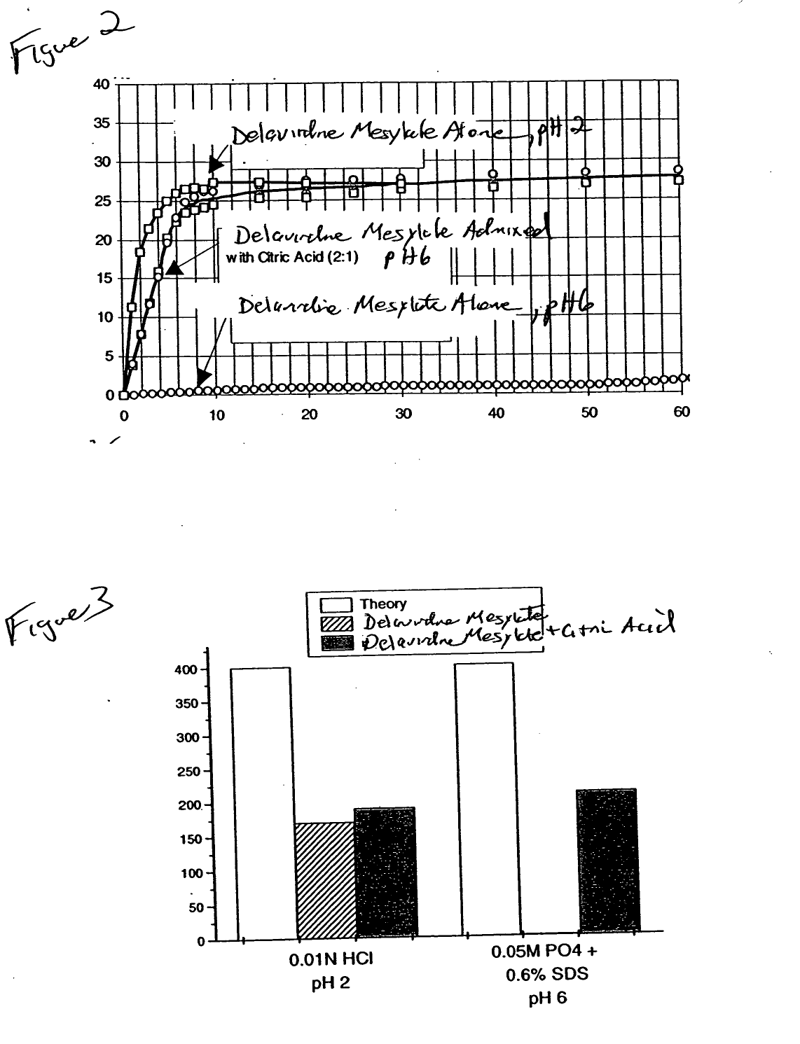

The quantitative intrinsic dissolution rates and the pH dependency of the intrinsic dissolution rates of the delavirdine mesylate-citric acid co-compressed admixture (2:1) are shown in FIG. 3 and the results are summarized in Table 1.

TABLE 1
Intrinsic Dissolution Rates of Delavirdine
Mesylate and Delavirdine Mesylate-Citric
Acid Admixture in Comparison with Theory.

| | IDR (micrograms · cm⁻² · sec⁻¹) | |
|---|---|---|
| Material | pH 2 0.01 N HCl | pH 6 with 0.5% SLS |
| Theory for Delavirdine | predicted ˜400^{a} | predicted ˜400^{a} |
| Mesylate | | |
| Delavirdine Mesylate | 220 | 2 |
| Observed | | |
| Delavirdine Mesylate-Citric | 190 | 160 |
| Acid (2:1) Admixture, | | |
| Observed | | |

^{a}Calculated using the following equation: J = D_{HA} · [HA]₀ · h⁻¹ + D_{H} · ([H⁺]₀ − [H⁺]ₕ) · h⁻¹ + D_{OH} · [OH⁻]ₕ − [OH⁻]₀) · h⁻¹

Thus, co-compression of delavirdine mesylate with citric acid prevents the surface precipitation of delavirdine free base and this is the reason for the rapid dissolution at pH 6. The dissolution of the pellets containing citric acid admixed with delavirdine mesylate showed almost no dependency on the bulk solution pH (Table 1), and there was no change in the bulk pH of the dissolution media.

Powder x-ray diffraction (XRD) analysis of a delavirdine mesylate pellet after dissolution at pH 2 showed the spectrum of anhydrous crystalline delavirdine free base (FIG. 4) indicating that transformation of delavirdine mesylate to the delavirdine free base occurred on the surface of the pellet during dissolution.

Based on the appearance of a pellet of delavirdine mesylate alone and the delavirdine mesylate-citric acid admixture (2:1) after dissolution under the microscope along with the XRD analysis data.

A proposed mechanism for the appearance of delavirdine free base on the surface of the salt is a follows. According to theory, without buffer, a highly concentrated solution of delavirdine mesylate is generated at the delavirdine mesylate crystal-liquid surface, with a concentration of at least 200 mg/mL. This surface solution of delavirdine mesylate is highly supersaturated with respect to the free delavirdine, since the pH is 2.88 (uncorrected for ionic strength), which is believed to be too high to maintain the solubility of delavirdine free base. As a result, precipitation of delavirdine free base should occur. However, delavirdine free base is precipitated as an oily form directly on the surface of the dissolving delavirdine mesylate, as evidence of coalescence on the surface of the pellet can be seen under a microscope. The oily free base probably undergoes surface diffusion, sintering (see Ristic', “Sintering—New Developments” in Materials Science Monograph 4, Elsevier Scientific Publishing Co. (New York, 1997)), and crystallization, resulting in crystalline delavirdine free base trihydrate on the surface of the pellet as established by x-ray diffraction. The dissolution rate is markedly reduced due to a contiguous film of crystalline delavirdine free base that is formed on the surface of the delavirdine mesylate pellet.

Examination of the pellets under the microscope immediately after dissolution showed that the oily particles (delavirdine) were weakly birefringent whereas, the material (delavirdine) on the outer surface of the pellet appeared to be birefringent.

Also, the delavirdine mesylate-citric acid (2:1) co-compressed admixture may not result in precipitation of delavirdine free base on the surface of the dissolving pellet due to the lower surface pH. The lower surface or diffusion layer pH results in a lower degree of supersaturation with respect to delavirdine free base, thereby preventing precipitation of the free base. This fact accounts for the remarkably fast dissolution of the delavirdine mesylate-citric acid admixture at pH 6.

In conclusion, the intrinsic dissolution rate of delavirdine mesylate is rapid at pH 1-2, but dissolution is slow at pH 6 due to the rapid conversion to delavirdine free base on the surface of the pellet during dissolution. This is the reason why the intrinsic dissolution rate is slow at pH 6. The intrinsic dissolution of the delavirdine mesylate-citric acid (2:1) admixture, however, is approximately 200 times faster than that of delavirdine mesylate alone because the lower pH of the aqueous diffusion layer prevents the surface precipitation of the free base. The delavirdine mesylate-citric acid admixture might be advantageous by showing a higher oral bioavailability than that of the mesylate salt, especially at a high stomach pH.

Intrinsic Dissolution of the Delavirdine Mesylate-citric Acid (2:1) Admixture

This study shows that the delavirdine mesylate-citric acid (2:1) admixture co-compressed with the Carver press produced a large increase in the intrinsic dissolution rate at pH 6 with 0.6% SLS. The intrinsic dissolution rate of the delavirdine mesylate-citric acid admixture is approximately 100 times faster than that of pure delavirdine mesylate alone (Table 1, FIGS. 2 and 3). Interestingly, the dissolution rate at pH 6 is surprisingly fast, and it is similar to that at pH 2.

The delavirdine mesylate-citric acid (2:1) admixture is completely dissolved in the pH 2 and the pH 6 dissolution fluid containing 0.6% SLS, whereas, delavirdine mesylate alone, is only approximately 60% dissolved at pH 2. Thus, the delavirdine mesylate-citric acid admixture prevents the precipitation of the free base on the surface of the dissolving salt at both pH 6 as well as at pH 2.

### Example 2

### Roller Compaction and Dissolution of a (2:1) Co-compressed Admixture of a Soluble Salt of a Poorly Soluble, Basic Drug and an Acidic Excipient

Materials and Methods

Delavirdine mesylate is a soluble salt of the poorly soluble, basic drug delavirdine, which can be prepared as described, for example, in PCT International Publication No. WO91/09849 (Romero et al.). Tablets including delavirdine mesylate (e.g., 100 mg or 200 mg) are available under the trade designation RESCRIPTOR from Pfizer Inc., New York, N.Y. Citric acid monohydrate is an acidic excipient that is available from Mallinckrodt, Hazelwood, Mo.

Roller Compaction of Delavirdine Mesylate with Citric Acid

Roller compaction was conducted using a Vector TF-Mini roller compactor with smooth, DP type rolls. The ingredients used for the compaction were weighed and screened using a #30 mesh screen. The ingredients were then hand mixed and added to the hopper of the roller compactor. The powder was granulated using a roll pressure of approximately 3 tons and a hopper feed-screw speed of 7 rpm.

The roll speed was determined as the speed that would produce an acceptable ribbon that would not bog down the compactor, which resulted in approximately 5-7 rpm. The ribbon produced was then fed through a conical mill (Quadro Comil, Model 197S) with a round screen (#2A-062R037/41), a standard impeller (#2A-1601-173), and a 0.38 cm (0.150 inch) spacer.

If smaller granules were desired, the mix was passed through the Comil a second time using a smaller round screen (#2A039R031/25). The granules were then screened to remove large granules and fines as would typically be done in a roller compaction process. Screens with #18 and #140 mesh were used to remove granules larger than 1000 micrometers and smaller than 105 micrometers, with the remainder used for further testing. Typically the granules removed at this point would be recycled back into the roller compactor, but this was not done in this case to avoid any possible effect on the granule properties that reworking might cause. The lots prepared for the roller compaction study with the ingredients used for each lot are shown in Table 2.

It was apparent from preliminary studies that roller compaction of delavirdine mesylate and citric acid was more convenient with the addition of other excipients due to the lack of cohesiveness and excessive sticking to the rolls when the mixture was used alone. Further experiments were therefore conducted to identify excipients that could be added to improve the processing characteristics of the mixture without adversely affecting the dissolution rate.

Roller compaction was attempted using the drug/citric acid mixture with addition of microcrystalline cellulose (Avicel) to improve the cohesiveness of the mixture. This produced a marginally acceptable ribbon, but sticking to the rolls again limited the utility of this method. When a granulation was attempted with microcrystalline cellulose (e.g., available under the trade designation Avicel) and magnesium stearate (0.5%), an acceptable ribbon was produced that was easily milled to produce granules. The delavirdine-citric acid granules were produced with either Avicel or Avicel and magnesium stearate, and these granules were used for further dissolution testing.

It was found that the addition of magnesium stearate slowed the dissolution of delavirdine mesylate relative to the granules with Avicel alone and, therefore, the addition of magnesium stearate was avoided in subsequent experiments.

TABLE 2
Lots Prepared (#31610-JMH-X) for Roller Compaction
Studies and Ingredient Amounts (gm)

| | JMH- | JMH- | JMH- | JMH- | JMH- |
|---|---|---|---|---|---|
| Ingredient (EDP #) | 004A | 004B | 009 | 010 | 001 |
| Delavirdine Mesylate | 253.3 | 136.5 | | 100.8 | 100.8 |
| Lot (B2)PART B- | | | | | |
| 4002 (99.2%) | | | | | |
| Delavirdine | | | 100.0 | | |
| Hydrochloride Lot | | | | | |
| (A)26162-MAL-32-B | | | | | |
| Citric Acid | 126.7 | 68.3 | 50.0 | | 50.0 |
| Anhydrous USP | | | | | |
| (216700) | | | | | |
| Microcrystalline | 76.0 | 51.2 | 30.0 | 110.0 | 30.0 |
| Cellulose, Avicel PH- | | | | | |
| 101 Bolted (154650) | | | | | |
| Lactose NF | | | 30.0 | 110.0 | 30.0 |
| Monohydrate Spray | | | | | |
| Process Standard | | | | | |
| (144630) | | | | | |
| Magnesium Stearate | | 1.28 | | | |
| NF Bolted (240857) | | | | | |

Avicel and lactose were investigated to determine the processability of the mixture and the effect on dissolution.

Granulations were conducted in identical fashion to those above, with the addition of the Avicel/lactose mixture to the delavirdine mesylate and citric acid. This combination produced an acceptable ribbon that could easily be milled to granules.

There was sticking seen using these excipients, however, and the method would likely be unacceptable for larger scale manufactures. A granulation with all of these ingredients was compared to a granulation prepared with no citric acid as a control experiment.

USP Dissolution Rate Determination

A dissolution test was conducted using tablets including delavirdine mesylate (e.g., 100 mg or 200 mg), available under the trade designation RESCRIPTOR from Pfizer Inc., New York, N.Y. The test utilized the USP 2 apparatus (paddle) operated at 50 rpm with 0.05 M pH 6.0 phosphate at pH 6, 0.6% sodium dodecylsulfate (SDS) in the dissolution medium. These conditions were chosen for examining the delavirdine mesylate-citric acid admixtures after investigating various pH and agitation conditions. The specified medium enhanced the formulation discriminating ability of the dissolution profiles due to the gradual slope of the curves.

Intrinsic Dissolution Rate Determination

The intrinsic dissolution rate of the delavirdine mesylate-citric acid (2:1) powdered solids was studied with the fiber optic dissolution apparatus. All experiments were conducted at 37° C. using either pH (0.01 N HCl) or 0.05 M phosphate buffer containing 0.6% SDS.

Results

Intrinsic Dissolution Rate Studies on Delavirdine Mesylate-citric Acid Granules Made by Roller Compaction

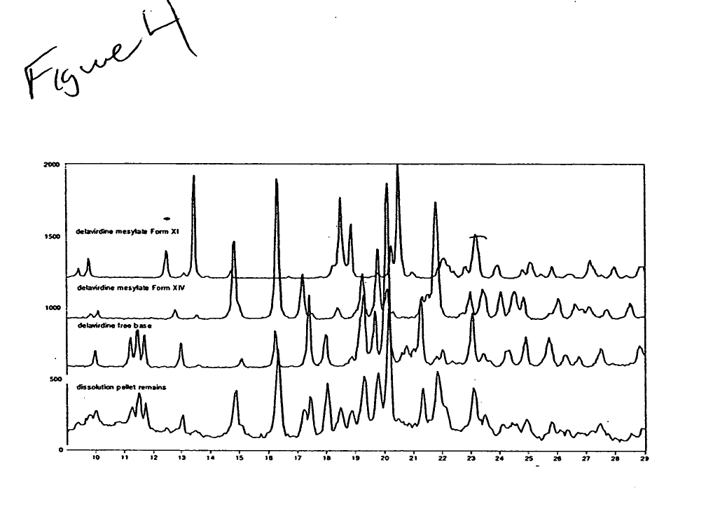

FIG. 5 shows the measured intrinsic dissolution rates using constant surface area pellets for two delavirdine mesylate-citric acid co-compressed admixtures prepared by roller compaction. Lots 31610-JMH-004a and JMH-004b both showed much faster intrinsic dissolution than delavirdine mesylate alone.

The only difference between lots JMH-004a and JMH-004b was the presence of magnesium stearate in lot JMH-004b. The presence of magnesium stearate appeared to decrease the dissolution rate performance somewhat.

The results for the roller compacted granules were consistent with those obtained for the cocompressed mixtures. In general, the intrinsic dissolution rate of the delavirdine mesylate in the granules was greater than that of delavirdine mesylate bulk drug at pH 2 suggesting that the pH of the diffusion layer was being reduced by the presence of citric acid.

To ensure that the acceleration in the dissolution was not caused by simply dispersing the drug with the citric acid, we ran the IDR experiment with a pellet of 2:1 delavirdine mesylate with lactose. In this case, the dissolution rate was approximately 50 times less than that of the delavirdine mesylate-citric acid granules. This experiment indicated that the dispersion of the drug was not important, but that modification of the pH within the diffusion layer surrounding the dissolving drug was the critical factor in the improved dissolution behavior of the admixture.

USP Dissolution Behavior of the Delavirdine Mesylate-citric Acid (2:1) Co-compressed Admixture as Granules

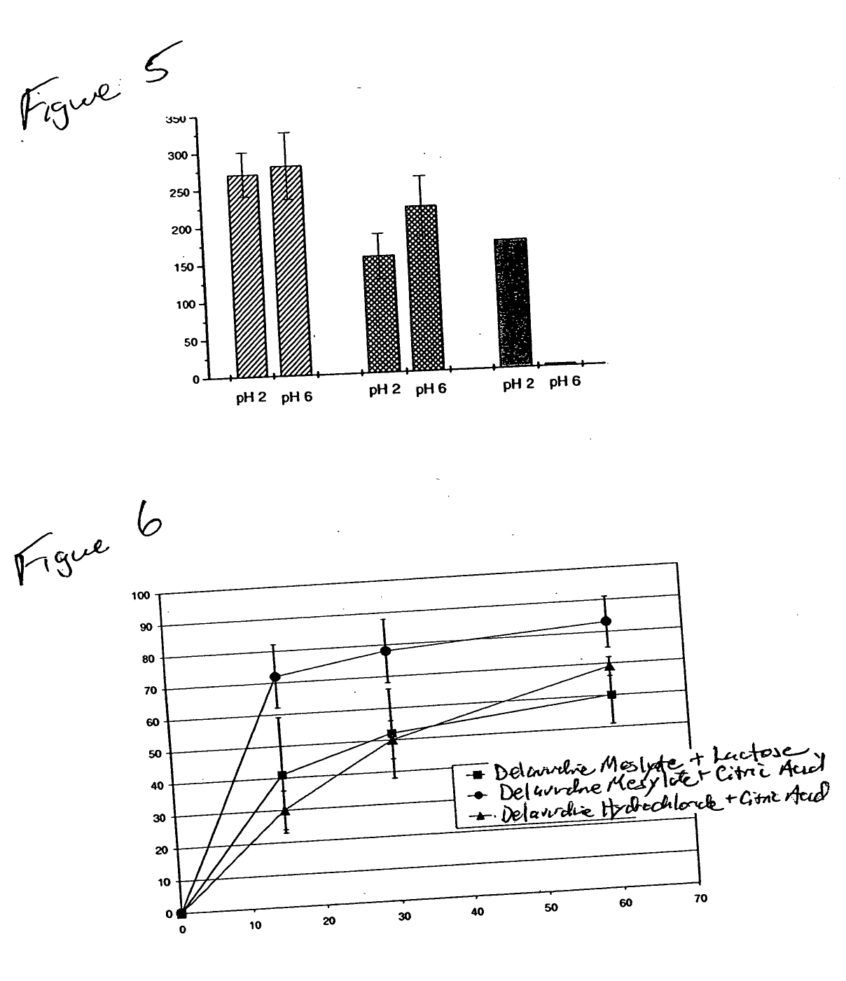

FIG. 6 shows the USP dissolution rates at pH 6 with 0.6% SLS for three different materials in a capsule measured at pH 6 with 0.6% SLS. These are delavirdine mesylate+lactose (2:1) granules as a control (JMH-010), delavirdine mesylate+citric acid (2:1) roller compacted granules (JMH-004a).

The data clearly shows that the delavirdine mesylate-citric acid granules dissolve very rapidly. Importantly, the dissolution rate was significantly improved over the delavirdine mesylate-lactose formulation. This agrees with the intrinsic dissolution rate results and suggests that the pH of the dissolving microenvironment is the important factor in determining the dissolution performance. Finally, the variability in the dissolution profiles of both of the citric acid formulations is less than that of the lactose formulation. This again agrees with our model of the behavior of the granules, since precipitation of the base (an inherently poorly reproducible process) is eliminated or reduced through the use of the citric acid.

Discussion

Based on the above analysis, diffusion layer pH modulated solids prepared with salts of ionizable drugs co-compressed or otherwise affixed to acidic or basic excipients offer the possibility of improving both the dissolution and the oral bioavailability of salts of poorly soluble drugs including the parent poorly soluble free acids and bases.

The dissolution rate at pH 6 with the delavirdine mesylate-citric acid co-compressed admixture is approximately 200 times faster than that of the delavirdine mesylate bulk drug alone at pH 6. This is attributed to the lower diffusion layer pH with the delavirdine mesylate-citric acid co-compressed admixture and this prevents surface precipitation of delavirdine free base and results in rapid dissolution even at pH 6.

### Example 3

### Bioavailability in the Rat of a Co-compressed Mixture of a Soluble Salt of a Poorly Soluble, Basic Drug and an Acidic Excipient

Materials and Methods

Delavirdine mesylate is a soluble salt of the poorly soluble, basic drug delavirdine, which can be prepared as described, for example, in PCT International Publication No. WO91/09849 (Romero et al.). Tablets including delavirdine mesylate (e.g., 100 mg or 200 mg) are available under the trade designation RESCRIPTOR from Pfizer Inc., New York, N.Y. Citric acid monohydrate is an acidic excipient that is available from Mallinckrodt, Hazelwood, Mo.

Rat Oral Bioavailability of Delavirdine Mesylate-citric Acid (2:1) Co-compressed Admixture Compared to a Delavirdine Mesylate Tablet

The oral bioavailabilities of a delavirdine mesylate-citric acid (2:1) co-compressed admixture and a 200 mg delavirdine mesylate tablet available under the trade designation RESCRIPTOR from Pfizer Inc., New York, N.Y., were determined in the rat (n=4) upon oral administration (intubation) of powdered (granular) forms of these two materials at a dose of 20 mg/kg. The rats (male, 360-400 gm) were surgically implanted with external jugular vein cannulas and they were allowed to recover for 1 week before use. The rats were fasted for 16 hours prior to dosing.

The delavirdine mesylate-citric acid (2:1) admixture was co-compressed at a pressure of approximately 255 MPa (37,000 psi) on a Carver press and the pellets were lightly ground with a mortar and pestle to give a coarse granule. This material was placed into one end of a 10 cm (4 inch) section of 0.48 cm (3/16 inch outside diameter)×0.16 cm (1/16 inch) inside diameter Teflon tube and the powder was held in place with a small amount of cheese (American, Fat Free). This tube, with the drug powder loaded in the distal end, was affixed to a 1 mL syringe and the tube was inserted into the stomach of the rat followed by administration of 1 mL of pH 5 (0.001 M) acetate buffer through the tube.

Blood samples (0.20 mL) were withdrawn from the jugular vein and placed in 1 mL lithium heparin test tubes. After centrifugation, the plasma was collected and stored at −20° C. until the time for assay. The plasma levels were determined by HPLC and the concentration of delavirdine (as free base equivalents) was determined using a series of plasma samples spiked with known amounts of delavirdine free base.

The plasma levels of delavirdine were determined by HPLC as described above. The concentrations were determined by the peak area method in comparison with a series of standards.

Results

The objectives of this study were to determine the oral bioavailability in the rat with at a stomach pH of 5, upon oral administration of the delavirdine mesylate-citric acid (2:1) admixture in comparison with that of a 200 mg tablet of delavirdine mesylate available under the trade designation RESCRIPTOR from Pfizer Inc., New York, N.Y. The dose of the delavirdine mesylate salt that was administrated orally in the rat was 20 mg/kg.

The following rat oral bioavailability study was conducted using a stomach pH of 5 in attempt to see if the delavirdine mesylate-citric acid admixture might have advantage in achlorhydrics, which is common in patients with acquired immunodeficiency syndrome (AIDS) (Zimmerman et al., Int. J. Clin. Pharmacol. Ther., 32:491-496 (1994)).

Rat Oral Bioavailability of Delavirdine Mesylate-citric Acid (2:1) Co-compressed Admixture Compared to a Delavirdine Mesylate Tablet at a Stomach pH of 5

The oral bioavailability of delavirdine mesylate-citric acid (2:1) co-compressed admixture was evaluated in the rat (n=4, 20 mg/kg) after oral administration at a stomach pH of 5 in comparison with that of a 200 mg delavirdine mesylate tablet available under the trade designation RESCRIPTOR from Pfizer Inc., New York, N.Y.

The bioavailability study was conducted by oral intubation of the delavirdine mesylate-citric acid (2:1) co-compressed admixture as a granular powder as well as a portion of the 200 mg delavirdine mesylate tablet as a granular powder by oral administration (intubation) of these two materials at a dose of 20 mg free base equivalents per kilogram (fbe/kg). Table 3 shows the concentration of delavirdine in the rat plasma as determined by HPLC. TABLE 3: Concentration of delavirdine in rat plasma after oral administration to rats (n=4) of a powdered 200 mg delavirdine mesylate tablet (e.g., a granular powder) available under the trade designation RESCRIPTOR from Pfizer Inc., New York, N.Y., and delavirdine mesylate-citric acid admixture (2:1) co-compressed as a granular powder, both dosed orally at 20 mg delavirdine mesylate fbe/kg.

Delavirdine Level in Plasma (micrograms/mL)

| | | Delavirdine Mesylate- |
|---|---|---|
| Time | Delavirdine Mesylate | Citric Acid (2:1) |
| (Hours) | Tablet, Powdered | Admixture, Powdered |
| 0.25 | 0.60 | 0.41 |
| 0.5 | 0.52 | 0.87 |
| 1 | 0.64 | 3.69 |
| 1.5 | 1.10 | 2.68 |
| 2 | 1.40 | 3.83 |
| 3 | 1.14 | 2.52 |
| 4 | 1.12 | 2.34 |
| 6 | 0.71 | 1.96 |
| 8 | 0.95 | 1.5 |
| 12 | 0.40 | 0.94 |
| 24 | 0.21 | 0.10 |

FIG. 7 shows a plot of the data and it is seen that the rat oral bioavailability of the delavirdine mesylate-citric acid (2:1) admixture is approximately 2 fold higher as estimated by AUC summation than that of a 200 mg delavirdine mesylate tablet available under the trade designation RESCRIPTOR from Pfizer Inc., New York, N.Y. (20 mg/kg, n=4) using a stomach pH of 5 (0.001 M), acetate buffer.

The data suggests that the increased bioavailability of the co-compressed delavirdine mesylate-citric acid (2:1) granular admixture is the result of the lower diffusion layer pH at the surface of the admixture which allows rapid and more complete dissolution of the drug.

Thus, the enhanced bioavailability of delavirdine mesylate-citric acid admixture in this rat study is probably due to the ability of the admixture (a) to rapidly dissolve despite the high bulk pH present in the rat stomach for these experiments, and (b) to form a supersaturated solution in the stomach and intestine.

Intrinsic dissolution rate studies have shown that at pH 5, delavirdine mesylate alone dissolves very slowly because a film of the free base forms very rapidly directly on the surface of the dissolving mesylate salt crystals. Once the free base forms on the surface, the bioavailability of delavirdine from that form is relatively low, because dissolution is inhibited. In the case of the co-compressed delavirdine mesylate-citric acid (2:1) granular admixture, however, the pH of the diffusion layer is kept low and, therefore, dissolution proceeds relatively fast and oral bioavailability is improved.

In conclusion, the oral rat bioavailability of the delavirdine mesylate-citric acid (2:1) co-compressed admixture is approximately 2-fold higher than that of the delavirdine mesylate tablet at a stomach pH of 5. This co-compressed diffusion layer modulated powdered admixture of delavirdine mesylate and citric acid in tablet or capsule form has the potential of generating higher and more uniform blood levels in AIDS patients since they typically have high stomach pH values.

Conclusions

The rat oral bioavailability at an initial stomach pH of 5, however, showed approximately a 2-fold higher bioavailability for the delavirdine mesylate-citric acid co-compressed powdered admixture as compared to the delavirdine mesylate tablet available under the trade designation RESCRIPTOR from Pfizer Inc., New York, N.Y. This indicates that the delavirdine mesylate-citric acid admixture should have the advantage of more uniform blood levels especially at high stomach pH values, typical of many AIDS patients.

### Example 4

### Preparation and Rat Oral Bioavailability of Spray Dried Powders of a Soluble Salt of a Poorly Soluble, Acidic Drug and a Basic Excipient

Background

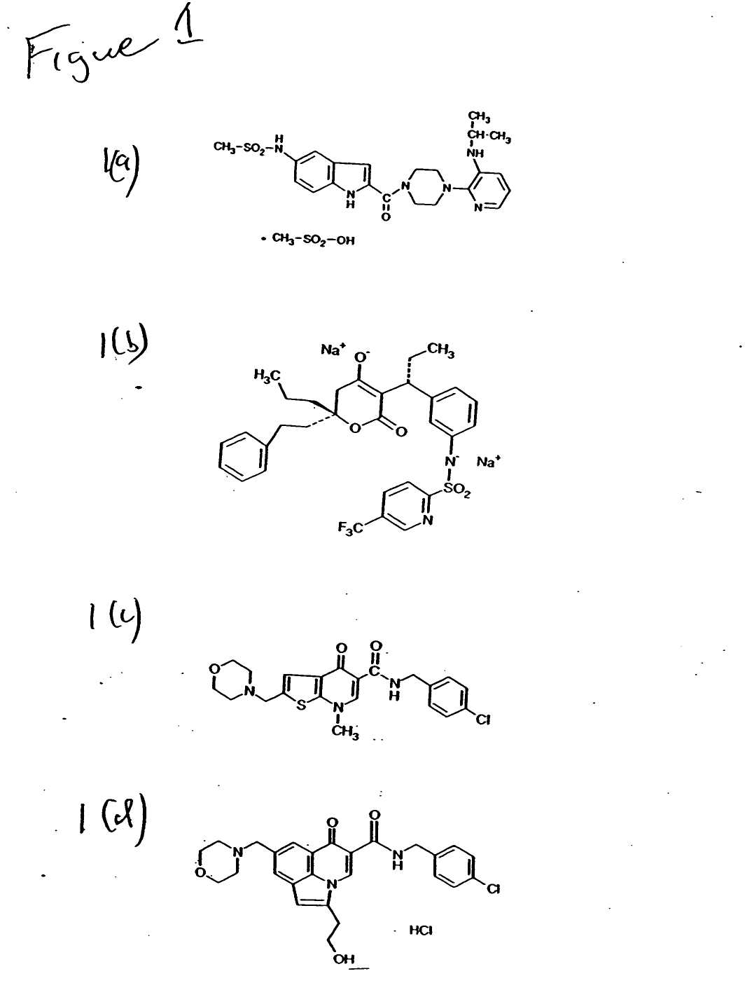

Tipranavir disodium (FIG. 1b), is the di-sodium salt of a poorly soluble, di-acidic drug (i.e., tipranavir) with a water solubility of approximately 5-10 micrograms/ml at pH 6. Low oral bioavailability observed with tipranavir disodium bulk drug in capsule formulations may be due to salt hydrolysis and precipitation of the corresponding free acid, tipranavir, in the stomach and intestine in-vivo.

This example is a demonstration of the preparation of spray dried powdered forms of tipranavir disodium containing basic excipients and polymers or surfactants, and the determination of the oral bioavailability in the rat.

Preparation of Ttipranavir Disodium Spray Dried Bulk Drug Powders

The bulk powders were prepared by spray drying aqueous solutions of tipranavir disodium along with various excipients. A summary of the spray dried formulations is presented in Table 4. A Yamato GA-21 lab scale spray dryer was used for all trials. Basic excipients used included polyvinylpyrrolidone (povidone, PVP; K-value 30). Additional excipients included Trehalose (a disaccharide sugar), hydroxy propyl methyl cellulose (HPMC; 2910, 3 centipoise), tris(hydroxymethyl)-aminomethane (TRIS or THAM), and a surfactant available under the trade designation PLURONIC F68 (available from BASF, Mt. Olive, N.J.).

The drug/excipient solutions were spray dried in the Yamato spray dryer using nominal inlet and outlet temperatures of 125° C. and 70° C., respectively (Table 4). The spray dry rate was 2.5-5 g/minute, atomization was 0.5-1 bar, and airflow 3.5-4.0 TFM. The yellow, free flowing powders were removed from the cyclone, placed in Teflon lined glass screw-top vials, and stored under refrigerated conditions. Yields of 50-85% of theory were obtained, which is typical for this spray dryer unit.

The bulk powders were isolated by spray drying and subsequent collection within the Yamato GA-21 cyclone. Yields of 50-85% were obtained, which is typical for this spray dryer unit. HPLC analysis confirmed that the neither the drug nor the excipients were preferentially lost; that is, potency was very close to theoretical once water content was accounted for.

For some of the samples, an additive such as sodium lauryl sulfate (SLS) was blended into the spray dried powder as indicated in Table 4.

TABLE 4
Composition of tipranavir disodium spray dried powders.

| Title | Composition of Spray Dried Powder |
|---|---|
| Tipranavir Disodium | Tipranavir Disodium 28.5 g |
| Tipranavir Disodium/ | Tipranavir Disodium 28.5 g and THAM 2.67 g |
| THAM | |
| Tipranavir Disodium/ | Tipranavir Disodium 28 g, THAM 2.67 g, and |
| THAM/F68 | F68 2.67 g |
| Tipranavir Disodium/ | Tipranavir Disodium 19 g, THAM 1.78 g, and |
| THAM/Trehalose | Trehalose 19 g |
| Tipranavir Disodium/ | Tipranavir Disodium 28.5 g, THAM 2.67 g, |
| THAM/HPMC | and HPMC 2.85 g |
| Tipranavir Disodium/ | Tipranavir Disodium 28.5 g, THAM 2.67 g, |
| THAM/PVP | and PVP 2.85 g |
| Tipranavir Disodium/ | Tipranavir Disodium 116.0 g and Trehalose |
| Trehalose | 40.0 g |
| Tipranavir Disodium/ | Tipranavir Disodium 116.0 g and HPMC 10.0 g |
| HPMC | |
| Tipranavir Disodium/ | Tipranavir Disodium 116.0 g and PVP 10.0 g |
| PVP | |

HPLC Analysis of Tipranavir in Rat Plasma Samples

HPLC analysis of tipranavir in the rat plasma samples following administration of the various tipranavir disodium spray dried powders was conducted using an RP 8 column (Zorbax, DuPont) with a mobile phase consisting of methanolaqueous 0.05 M formate buffer, pH 4 (75:27).

Rat Oral Bioavailability

The rat oral bioavailability of tipranavir disodium spray dried powders as well as the parent tipranavir disodium bulk drug were administered by intubation of the powders using a group of 7-8 rats (250-290 g) obtained from Taconic (Germantown, N.Y.). Intubation was achieved using a 10 cm (4 inch) section of Teflon tubing, 0.32 cm (⅛ inch) outside diameter×0.48 cm (3/16 inch) inside diameter, containing a piece of cheese (American, fat free) inserted into the bottom of the tubing. The desired tipranavir disodium powdered bulk drug was placed into the tube and the tube was inserted into the stomach of the rat. The drug was displaced from the Teflon tubing and into the stomach by passing 2 ml of water through the tubing. The dose was 20 mg/kg in all cases.

The blood samples were processed with precipitation of the proteins with acetonitrile followed by centrifugation. The samples were assayed as described above.

Rat Oral Bioavailability Studies

The rat oral bioavailability of tipranavir disodium powders (20 mg/kg) was calculated from the blood level curves shown in FIG. 2 and the AUC_{Inf} values are shown in Table 5.

TABLE 5
Comparison of the AUC_{Inf} Values in Rat Oral
Bioavailability Study with Tipranavir Disodium Spray
Dried Powders Dosed at 20 mg/kg.

| Spray Dried Bulk Drug | State | AUC_{Inf}^{b} |
|---|---|---|
| Tipranavir Disodium bulk | fasted | 23.4 micrograms · ml⁻¹ · hour |
| drug | | |
| Tipranavir Disodium + THAM + | fasted | 29.6 micrograms · ml⁻¹ · hour |
| HPMC | | |
| Tipranavir Disodium + THAM + | fed | 42.5 micrograms · ml⁻¹ · hour |
| PVP + SLS | | |
| Tipranavir Disodium bulk | fed | 45.8 micrograms · ml⁻¹ · hour |
| drug | | |
| Tipranavir Disodium + THAM + | fasted | 46.4 micrograms · ml⁻¹ · hour |
| PVP + SLS | | |

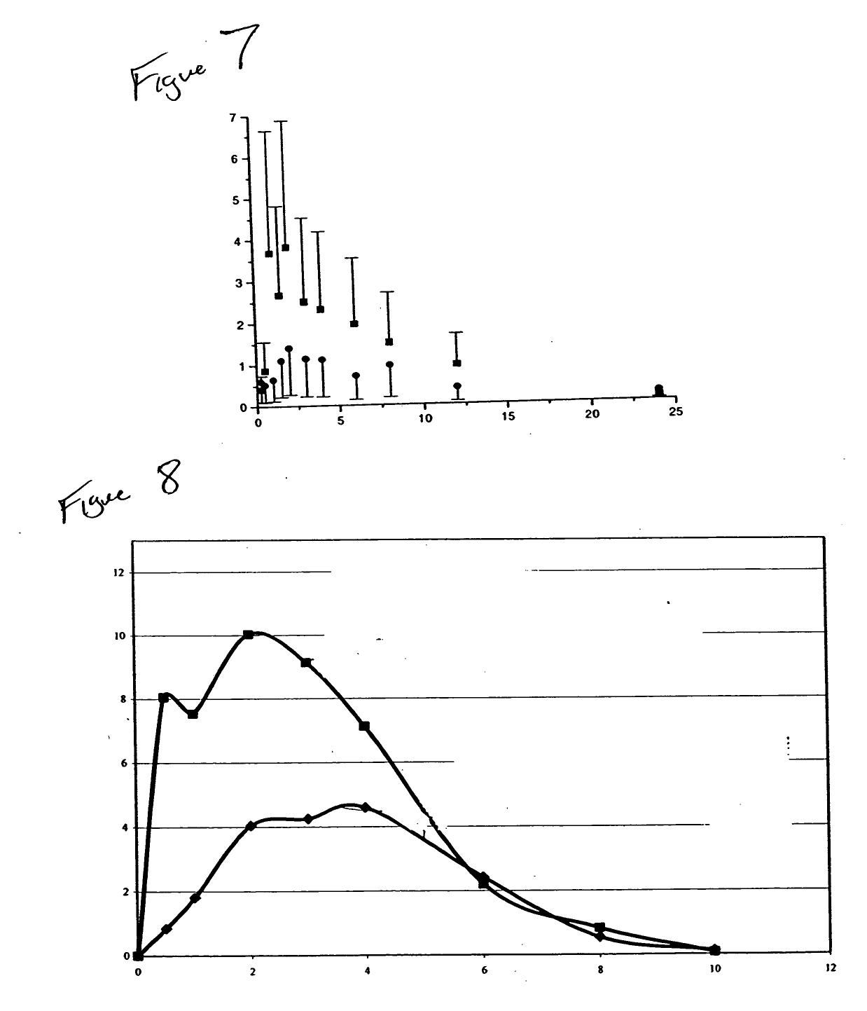

a. AUC Data taken from FIG. 8 using Win Nonlin.

Rat Oral Bioavailability of Tipranavir Disodium Spray Dried Powders

The rat oral bioavailability of tipranavir disodium spray dried powders (20 mg/kg) (FIG. 8 and in Table 5) showed that the AUC values was the highest (AUC=46.4 micrograms·ml⁻¹·hour) for the spray dried powder consisting of tipranavir disodium+THAM+PVP+SLS. The AUC of the latter was approximately 2 fold higher than that of the parent compound, tipranavir disodium (23.4 micrograms·ml⁻¹·hour, in the fasted state) whereas, in the fed state, the bioavailabilities were similar.

### Example 5

### Oral Bioavailability in Male Beagle Dogs of Formulations of a Soluble Salt of a Poorly Soluble, Basic Drug

Introduction

The poorly soluble, basic drug illustrated in FIG. 1c is a weak base with a pKₐ of 5.4. The intrinsic solubility on the poorly soluble, basic drug illustrated in FIG. 1c is less than 1 microgram/ml. The hydrochloric acid salt of the poorly soluble, basic drug illustrated in FIG. 1c is considered preferable to the free base as it is more soluble and has been shown to give better oral bioavailability in the rat at doses greater than or equal to 100 mg. In a subsequent dog study the oral bioavailability for the HCl-salt suspension was relatively low (27%) compared to a solution (97%). In another study in dogs, pretreatment with omeprazole (to raise stomach pH) and coadministration of an acid chaser was compared. It was found that the oral bioavailability of the poorly soluble, basic drug illustrated in FIG. 1c was significantly lower when the drug was given after pretreatment with omeprazole, where the stomach pH should be pH 4 to 6, than when given followed by an acid chaser, where the pH of the stomach should be pH 1 to 2. It was therefore concluded that the low oral bioavailability of the hydrochloride salt of the poorly soluble, basic drug illustrated in FIG. 1c in dogs was due to the high gastric pH in some individuals. It is hypothesized that high pH causes the drug to precipitate as the free base. Therefore, the oral bioavailability is reduced in those individuals with high stomach pH.

An option to solve this problem is to formulate solid particles, consisting of the drug co-compressed with an acid chosen to control the diffusion layer pH surrounding the dissolving co-compressed hydrochloride salt of the poorly soluble, basic drug illustrated in FIG. 1c granule. The acid is intended to maintain a low pH in the diffusion layer surrounding the granules, thereby achieving a high concentration of drug during dissolution. These diffusion layer pH modulated solids should prevent or decrease precipitation into the free base form (i.e., the poorly soluble, basic drug illustrated in FIG. 1c).

Formulations

HCl-salt aqueous suspension. The hydrochloride salt of the poorly soluble, basic drug illustrated in FIG. 1c was suspended in 0.15 M NaCl with 2% Cremophor EL to a concentration of 30 mg/g.

Preparation of the hydrochloride salt of the poorly soluble, basic drug illustrated in FIG. 1c co-compressed pH-modulated solid. The diffusion layer pH modulated solid form consisting of the hydrochloride salt of the poorly soluble, basic drug illustrated in FIG. 1c and citric acid was made in the following manner.

(1) The bulk hydrochloride salt of the poorly soluble, basic drug illustrated in FIG. 1c and citric acid were both hand-ground in a mortar and pestle.

(2) The ground materials were physically mixed in a 2:1 mass ratio, 2 grams of the hydrochloride salt of the poorly soluble, basic drug illustrated in FIG. 1c, Form I (34563-DCS-005) and 1 gram of citric acid.

(3) The mixture was slugged using a punch and die assembly, 0.64 cm (8/32 inch) with 9000 newtons (2000 pounds force). Tablets of approximately 100 mg each were made by co-compressing the hydrochloride salt of the poorly soluble, basic drug illustrated in FIG. 1c with citric acid. The inside of the punch and die assembly was coated lightly with sodium stearoyl fumarate to keep it from sticking.

Tablets were then prepared by lightly hand-grinding the co-compressed hydrochloride salt of the poorly soluble, basic drug illustrated in FIG. 1c-citric admixture in a mortar and pestle to produce a course powder that was filled into hard gelatin capsules #00 (Torpac, Hanover, N.J.). The amount filled in each capsule (302-357 mg) was adjusted to the weight of the dogs in order be equivalent to 15 mg/kg of free base.

Characterization of pH-modulated Hydrochloride Salt of the Poorly Soluble, Basic Drug Illustrated in FIG. 1c-citric Acid Co-compressed Admixture by Rotating Disk Dissolution.

The diffusion layer pH modulated solid was evaluated using rotating disk dissolution apparatus at pH 4 and 37° C., conditions under which a large depression in the dissolution rate of the hydrochloride salt of the poorly soluble, basic drug illustrated in FIG. 1c had already been observed with the pure drug alone. Detection of the drug was achieved using UV absorbance at 306 nm.

Animal Protocol—General Description

The formulations above were administered to 4 male Beagle dogs (Marshall Farms, USA, Inc., North Rose N.Y.). A one-week washout period was allowed between each administration. The dose was equivalent to 15 mg/kg of free base (i.e., the poorly soluble, basic drug illustrated in FIG. 1c). Control of gastric pH was provided by pretreatment with of 2×10 mg omeprazole (Prilosec, Astra Zeneca), given at approximately 18 hours and 1 hour prior to dosing of the test formulation.

The animals were weighed the morning before dosing and the dosage (15 mg free base equivalent/kg) and the corresponding volume or weight of the formulation was then calculated. Liquid formulations were administered by syringes that were weighed before and after administration. The dry formulation was weighed directly into hard gelatin capsules.

Blood samples (2 ml) were collected from the jugular vein or cephalic vein into EDTA vacutainer tubes at before dosing, and at 0.33, 0.67, 1, 2, 4, 6, 8, 12, and 24 hours after administration of the dose. Samples were stored up to 1 hour on ice before the plasma was separated by centrifugation at approximately 2000×g for 10 min. The separated plasma was collected in polypropylene storage vials and stored at −10° C. or colder until analyses.

Animal Protocol—Test System

The dogs were 1-5 years of age and they weighed 12-17 kg. The animals were individually identified by the use of ear tattoos. The animals did not have any apparent health abnormalities. Prior to initiation of the test, blood samples were submitted to a clinical lab for evaluation of complete blood chemistry and clinical chemistry.

The animals were housed in stainless steel cages with Aspen wood shavings for bedding. The Temperature was 65°-78° F. and the relative humidity 30-70%. Ventilation was greater than or equal to 12 changes/hour and fluorescent lighting on a 12 hour on/off cycle was provided.

The animals were fasted from the evening the day before and until 4 hours after dosing. Otherwise up to 400 g/day of PMI Certified Canine Diet #5007 was provided. Potable rechlorinated deionized water was provided ad libitum.

Determination of Drug Concentration in Plasma

The analytical method for determination of the poorly soluble, basic drug illustrated in FIG. 1c in dog plasma samples was based on LC-MS. Briefly, the method employed acetonitrile precipitation of plasma protein, a rapid separation of analytes on a C8 column in reversed-phase mode, and detection of analytes by positive ion atmospheric pressure chemical ionization (APCI-MS) with selected ion monitoring (SIM). The poorly soluble, basic drug illustrated in FIG. 1c was detected at an m/e of 432, corresponding to the M+H ion. The internal standard (IS) was detected at an m/e of 446. Signal intensity-time data were acquired and analyzed by the UPACS chromatography data system. The UPACS chromatography system identified baselines and performed peak area (PA) calculations. The peak area ratio (PAR) of the poorly soluble, basic drug illustrated in FIG. 1c versus the IS was calculated, and the instrument response was calibrated by linear regression analysis, weighted by 1/concentration, of the PAR versus the theoretical concentration of calibration standards prepared in the matrix. Plasma concentrations of study samples and QC samples were determined from the response calibration line.

Pharmacokinetic Calculations.

Concentration-time data for individual animals was compiled from both assays in the ADME database, which computed non-compartmental pharnacokinetic parameters from concentration-time profiles. In these calculations concentrations reported as “q,” that is, below the limit of quantitation, were treated as zeroes (Glass et al., ADME User's Manual, Version 5.0, Oct. 14, 1999).

The apparent terminal rate constant, λ_{z}, was by linear regression analysis of the terminal linear segment of semi-log transformed concentration-time data. The area under the plasma concentration-time curve from time zero to infinity, AUC_{0-∞}, was calculated as AUC₀₋ₜ+Ct/λ_{z}, where AUC₀₋ₜ is the area under the plasma concentration-time curve from time 0 to the last measurable plasma concentration, Cₜ, and λ_{z} is the apparent terminal rate constant. AUC₀₋ₜ was calculated by the method of linear trapezoids.

The observed maximum plasma concentration, Cₘₐₓ, and the time of its occurrence, tₘₐₓ, were determined by inspection of the concentration-time data. Means and standard deviations for AUC_{0-∞} and Cₘₐₓ were computed by hand.

Results

Characterization of Formulations—pH-modulated Solid

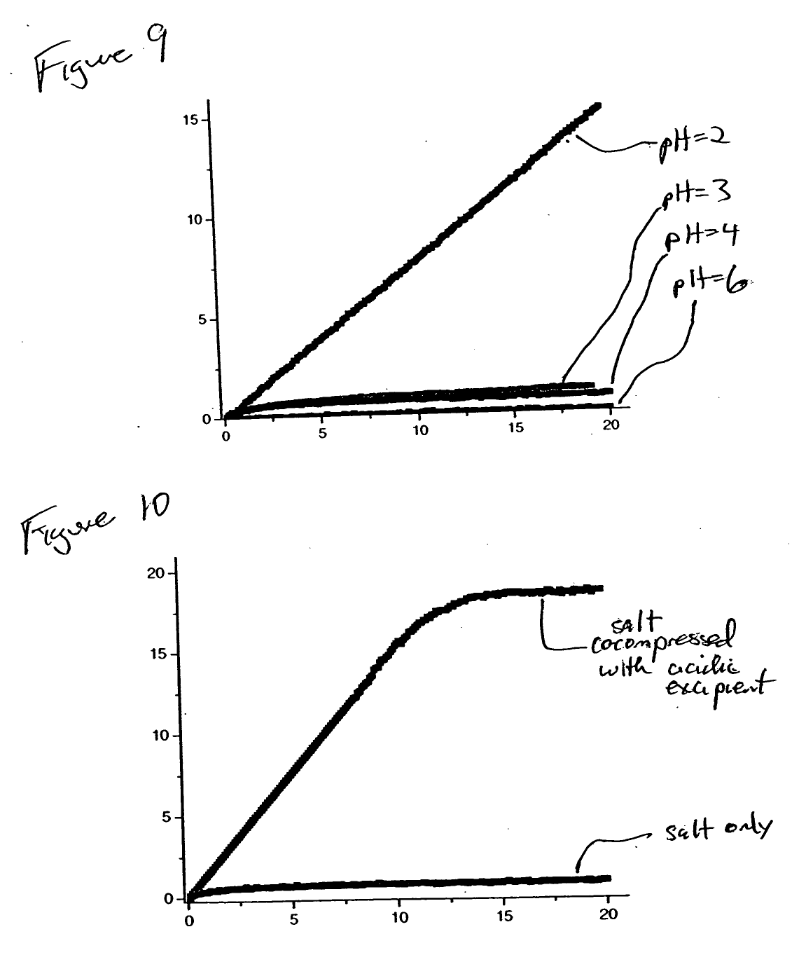

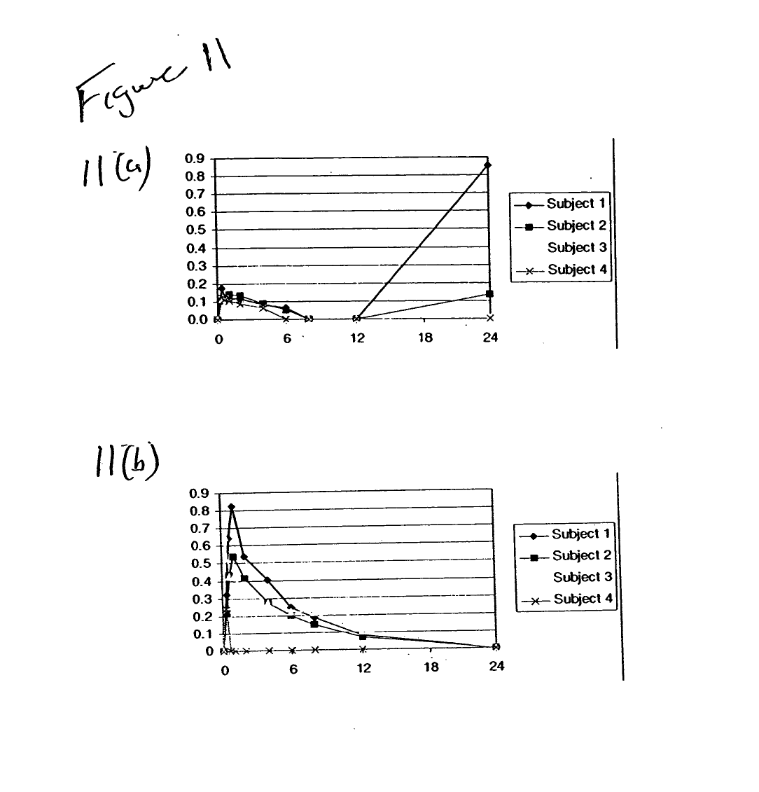

The diffusion layer pH modulated solid was characterized by measuring the dissolution performance using the rotating disk method at pH 4. FIG. 9 shows the measured rotating disk dissolution for the hydrochloride salt of the poorly soluble, basic drug illustrated in FIG. 1c as a function of pH. The dissolution rate rapidly decreased as the pH was increased, correlating with the observed low bioavailability in dogs with high pH stomachs. FIG. 10 shows the rotating disk data for the diffusion layer modulated solid. The dissolution rate showed a huge enhancement at pH 4 for the pH modulated solid with respect to the unbuffered bulk drug. The dissolution was so rapid that the entire drug/citric acid pellet was dissolved in approximately 12 minutes.

Bioanalytical Assay Performance

Assays were performed in two runs and data was acquired and archived on the UPACS data system. AUC calculations were performed by the ADME database and data and results were archived by ADME.

A 10 point standard curve, prepared in the plasma matrix, was assayed at the beginning and end of each run. The initial replicates of standards 1-5 of Assay 2 were dropped due to a laboratory error, but the second set of the standards, injected at the end of the run, were acceptable. The high standard (70.7 microM) for Assay 2 was dropped due to unacceptable response, however, no study sample approached this concentration. Repeat assays because of truncation of the standard curve were not required.

For acceptable standards, the lower limit of quantitation was 0.0495 microM, for which the overall recovery was 103% and coefficient of variation (C.V.) was 12%. Higher concentration standards were determined with a lower C.V., ranging from 1-8%. The low QC sample, prepared at 0.0982 microM, was determined 8 times in Assay 1 and 6 times in Assay 2. Over both assays the measured concentration of this QC sample ranged from 80-110% of the theoretical value, with an overall recovery of 91±8%. The overall recoveries of the middle (14.7 microM) and high (47.7 microM) QC samples were 108±6% and 97±5%, respectively, and the overall recovery of all QC samples was 99±10%. The performance of the assays, based on calibration standards data and QC data, suggests that the assays were performed with sufficient accuracy and precision to allow the evaluation of the bioavailability of formulations tested in the study protocol.

Four samples were reassayed because the reported concentrations of these samples were not consistent with other concentration-time data in the profile. For Subjects 1 and 2, in both treatments A and D, the sample at 24 hours appeared to increase in relationship to the previous sample (C24>C12). For all four samples, reassay in duplicate confirmed the initial result. Because the reassay data was not employed in the analysis of bioavailability, the reassay report was not archived in ADME, although the assay report is archived with the raw data for the study.

Performance of Prototype Formulations of the Poorly Soluble, Basic Drug Illustrated in FIG. 1c in Beagle Dogs

All formulations were tolerated well by the animals. No emesis was observed.

Individual and mean plasma concentration profiles are shown in FIGS. 11a and 11b. In general, concentration-time profiles consisted of a single concentration maximum observed between 0.33 and 2 hours, followed by a steady decline of plasma concentrations. In most cases an apparent terminal rate constant could be estimated, allowing the calculation of AUC_{0-inf}. For dogs 1 and 2 in treatment A, the plasma concentration of the poorly soluble, basic drug illustrated in FIG. 1c at 24 hours appeared to increase rather than decline (C24>C12). In this case, the observed 24 hour time point was ignored in the calculation of AUC_{0-inf}. For dog 4, treatment B, the oral bioavailability was so low that only one plasma sample was quantifiable, thus, no AUC_{0-inf} was calculated. Plasma concentration at the last sampling time (24 hours) were significantly higher that at the previous sampling occasion (12 hours) for subject 1 and 2 for 2 formulations. These data points have been excluded when calculating AUC, Cₘₐₓ and tₘₐₓ. The bioavailability for subject 4 was below the quantitation limit for all time points except one after administration of a pH-modulated solid. Consequently, calculation of AUC was not possible.

AUC, Cₘₐₓ and tₘₐₓ for the investigated formulations are shown in Table 6. For comparison, some results from earlier studies have been included. The HCl-salt suspension (reference formulation) showed a low AUC which was comparable to what was observed for the same formulation co-administered with omeprazole in an earlier study. This is not surprising, since the same individual animals were used in that study as in the present study, and suggests that data could be compared between the studies.

The AUCs were significantly higher for the pH-modulated system (approximately four times) than for a HCl-salt suspension with omeprazole co-administration.

Cₘₐₓ varied between formulations as described for AUC above. No clear differences in tₘₐₓ were observed.

TABLE 6
Results from administration of prototype formulations
of the poorly soluble, basic drug illustrated in FIG. 1c.
Standard deviations are shown within brackets.

| Formulation | | | | |
|---|---|---|---|---|
| of the poorly | | | | |
| soluble, basic | | AUC | | |
| drug illustrated | Premedication/ | (microM- | Cₘₐₓ | tₘₐₓ |
| in FIG. 1c | Coadministration | hour) | (microM) | (hour) |
| Suspension | Omeprazole | 0.79 (0.14) | 0.14 (0.03) | 0.33-1 |
| of the | (2 × 10 mg) | | | |
| hydrochloride | | | | |
| salt | | | | |
| Diffusion layer | Omeprazole | 3.58 (0.51) | 0.56 (0.25) | 0.33-1 |
| modulated | (2 × 10 mg) | | | |
| solid | | | | |

Discussion

The results suggest that pH-modulated solids are useful for improving the bioavailability of the hydrochloride salt of the poorly soluble, basic drug illustrated in FIG. 1c in individuals with a high gastric pH.

### Example 6

### Dissolution Profiles for Mixtures of a Soluble Salt of a Poorly Soluble, Basic Drug with an Acidic Excipient as a Function of Compression

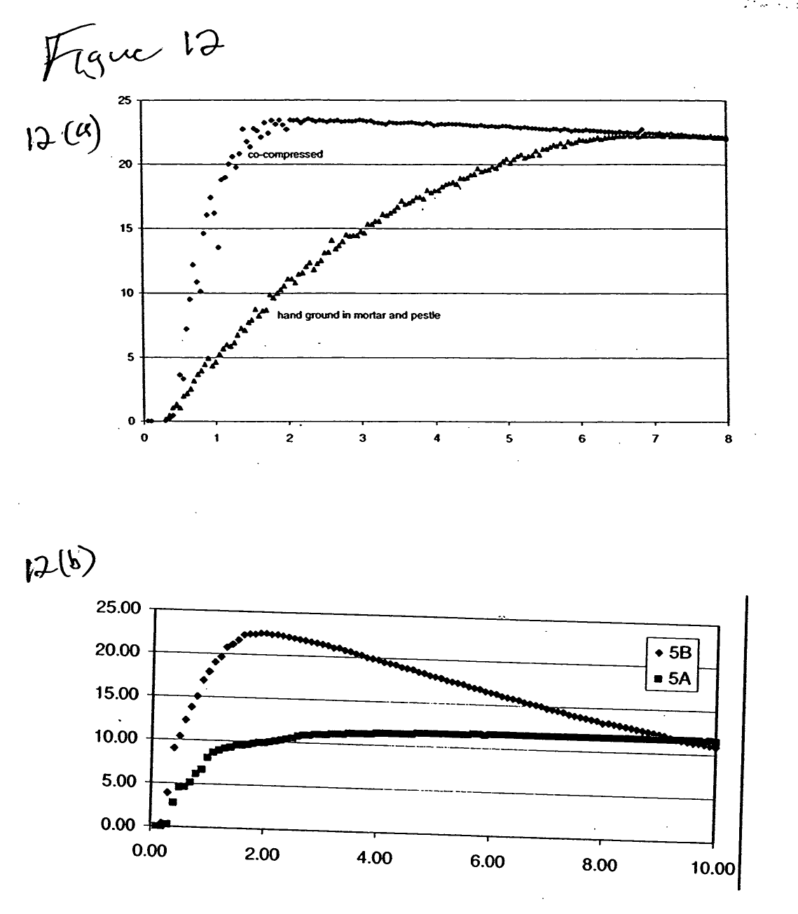

A delavirdine mesylate:citric acid 2:1 (w:w) admixture was co-compressed in a Carver press using a 0.48 cm (3/16 inch) punch and die combination at 255 MPa (37,000 psi) for one minute. A simple physical mixture of delavirdine mesylate:citric acid 2:1 (w:w) was also prepared by hand grinding the mixture in a mortar and pestle. The dissolution profiles in a pH 6 (0.05M phosphate) solution for the co-compressed mixture and the simple physical mixture were determined by measuring the concentration of delavirdine (micrograms/ml) as a function of time (minutes) as depicted in FIG. 12a. Dissolution of the co-compressed diffusion layer modulated (DLM) powder is far more rapid than the hand ground physical mixture of the two excipients.

Similarly, samples were prepared from a mixture of delavirdine mesylate:citric acid:lactose (2:1:1 w/w/w). Sample 5A was hand ground and placed as a powder in a dissolution basket. Sample 5B was co-compressed in a Carver press using a 0.48 cm (3/16 inch) punch and die combination at 255 MPa (37,000 psi) for one minute, and then lightly hand ground and placed as a powder in a dissolution basket. FIG. 12b illustrates a dissolution profile for the delavirdine mesylate co-compressed diffusion layer modulated solid (5B) as compared to a hand ground physical mixture of the components (5A) in a dissolution basket at pH 6 and 25° C. The diffusion layer modulated solid exhibits more rapid dissolution and also shows the ability to generate a solution of higher concentration than the mixture of the components alone. Dissolution rates similar to those observed for the sample co-compressed at 255 MPa (37,000 psi) were also observed for a sample that was co-compressed at 17 MPa (2500 psi).

Another experiment was designed to compare the bioavailability performance of a diffusion layer modulated solid to a mixture of the two excipients without co-compression using powder in a gelatin capsule. A diffusion layer modulated solid was formed from a 2:1 weight ratio of delavirdine mesylate and citric acid by co-compression in a Carver press using a 0.48 cm (3/16 inch) punch and die combination at 255 MPa (37,000 psi) for one minute. A hand ground physical mixture of delavirdine mesylate and citric acid in the same ratio was also prepared and placed into a gelatin capsule. The dissolution rate of the DLM solid was 3.04 mg/minute compared to 1.04 mg/minute at pH 6 for the simple physical mixture. Clearly, the dissolution rate of the DLM solid was enhanced by approximately three-fold with respect to a simple dry physical mixture of the two components.

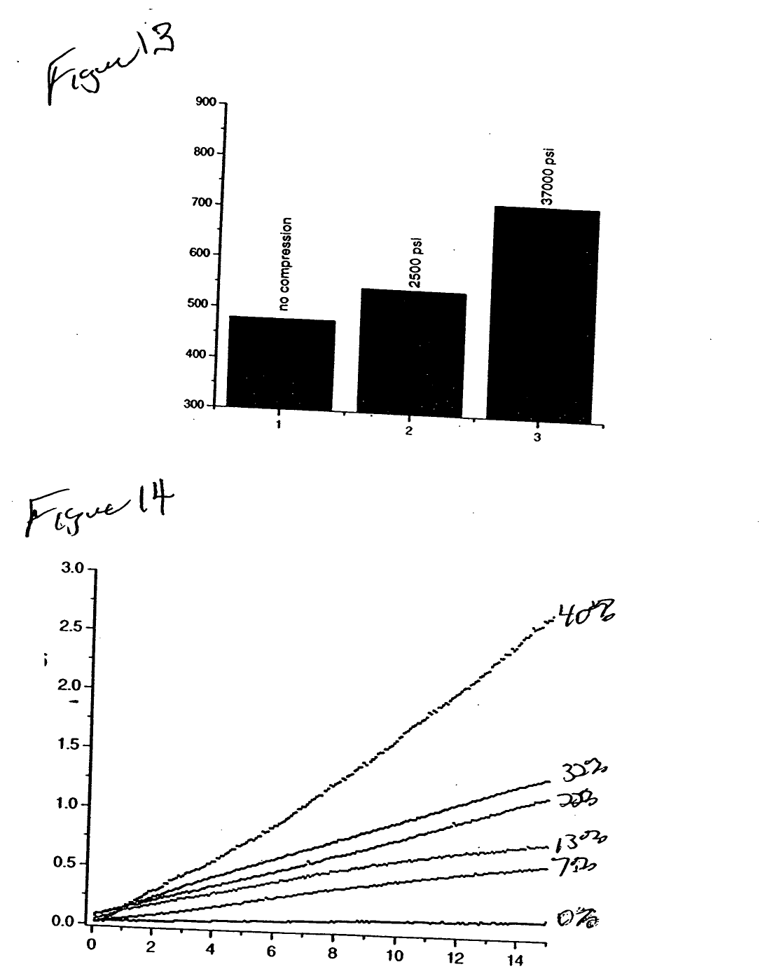

In another experiment, mixtures of 1:1 delavirdine mesylate:citric acid mixtures (w:w) were prepared. Samples of powders without compression, after compression at 17 MPa (2500 psi), and after compression at 255 MPa (37,000 psi) were placed in placed in capsules, and the relative dissolution rates in pH 6 media were determined as illustrated in FIG. 13. Dissolution rates were determined from the initial slope of the drug concentration vs. time profiles obtained after dissolution began. The data shows that the dissolution rate was fastest when the material was compressed at 255 MPa (37,000 psi). The material compressed at 17 MPa (2500 psi) showed only a slight enhancement in its dissolution rate with respect to the non-compressed material.

### Example 7

### Dissolution Profiles for Mixtures of a Soluble Salt of a Poorly Soluble, Basic Drug with an Acidic Excipient as a Function of Weight Fraction of the Acidic Excipient

Mixtures of the soluble hydrochloride salt (i.e., illustrated in FIG. 1d) of a poorly soluble, basic drug with an acidic excipient (e.g., malic acid) were prepared with 0-40% by weight malic acid. The mixtures were co-compressed in a Carver press using a 0.48 cm (3/16 inch) punch and die combination at 255 MPa (37,000 psi) for one minute and hand ground into a powder. A rotating disk procedure at 300 revolutions per minute (rpm), 25° C., and pH 6 (0.05M phosphate) was used to determine the dissolution profile by measuring the amount of sample dissolved (mg) over time (minutes). The dissolution profiles for the soluble hydrochloride salt illustrated in FIG. 1d-L-malic acid co-compressed admixtures are illustrated in FIG. 14. Significant enhancement in the dissolution rate was observed even at as low as 7% by weight of L-malic acid.

In another experiment, mixtures of a soluble salt (e.g., delavirdine mesylate) of a poorly soluble, basic drug (delavirdine) with an acidic excipient (e.g., citirc acid) were prepared in weight ratios of 1:7.5 (Sample A) and 1:1 (Sample B), delavirdine mesylate:citric acid. Sample B was co-compressed in a Carver press using a 0.48 cm (3/16 inch) punch and die combination at 255 MPa (37,000 psi) for one minute and then hand ground lightly into a coarse powder. Sample A consisted of the simple physical mixture of the drug (delavirdine mesylate) and the excipient (citric acid). The powders were placed in capsules and the dissolution rates were determined at pH 6. The dissolution rate of Sample A (the physical mixture) was 1.69 mg/minute, and the dissolution rate of Sample B (the co-compressed drug admixture) was significantly faster, 5.91 mg/minute. The dissolution rates were also determined at pH 2, with similar results: Sample A was 1.67 mg/minute and Sample B was 5.03 mg/minute. Thus, the diffusion layer modulated admixture dissolved faster than the simple physical mixture.

### Example 8

### Dissolution Profiles for Mixtures of a Soluble Salt of a Poorly Soluble, Basic Drug with an Acidic Excipient for Various Acidic Excipients

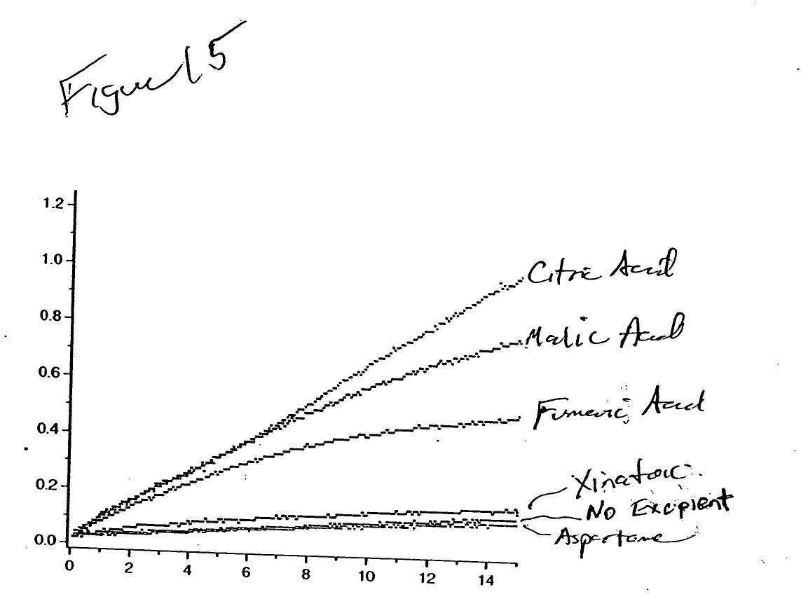

Mixtures of the soluble hydrochloride salt (i.e., illustrated in FIG. 1d) of a poorly soluble, basic drug with acidic excipients (e.g., citric acid, malic acid, fumaric acid, xinafoic acid, and aspartame) in approximately a 1:1 molar ratios were prepared. The mixtures were co-compressed in a Carver press using a 0.48 cm (3/16 inch) punch and die combination at 255 MPa (37,000 psi) for one minute, and the dissolution profiles were determined using a rotating disk procedure at 300 rpm, 25° C., and pH 6 (0.05M phosphate), by measuring the amount of the sample dissolved (mg) over time (minutes). The dissolution profiles for the mixtures are illustrated in FIG. 15. The highest dissolution rates were observed using fumaric acid, malic acid, and citric acid as the acidic excipient. The dissolution profile for the hydrochloride salt with no excipient is included in FIG. 15 for comparison.

### Example 9

### Microscopical Characterization of a Co-compressed Mixture of a Soluble Salt of a Poorly Soluble, Basic Drug with an Acidic Excipient

Light microscopy, Raman microscopy, and infrared microspectroscopy were used to compare two delavirdine mesylate:citric acid mixtures. One mixture was a roller compacted granulation at a pressure greater than 172 MPa (25,000 psi), and the other mixture was a lab scale, hand ground preparation made by grinding the two powders in a mortar and pestle fro one minute. As delavirdine mesylate:citric acid mixtures compacted in a Carver press using a 0.48 cm (3/16 inch) punch and die combination at 17 MPa (2,500 psi) did not stick together, no further microscopical characterization was performed on this sample. The analyses revealed significant differences in particle size and uniformity. The analyses revealed that roller compacted material is composed of large granules of finely blended components, while lab scale hand ground material was composed of unassociated, discrete heterogeneous particles. Raman and infrared microspectroscopical data revealed that hand ground material exhibited heterogeneity at approximately 100 micrometers spatial domain, whereas roller compacted material was relatively homogeneous down to approximately 15 micrometer spatial domains.

Light microscopy: Samples were examined with top/transmitted light using a stereomicroscope at 7× to 40×magnification available under the trade designation SMZ-10 (#AN079059) and a polarized light microscope (PLM) at 100-400×magnification available under the trade designation OPTIPHOT (#231561), all available from NikonUSA (Melevile, N.Y.).

Raman spectroscopy: A dispersive Raman microscope available from Thermo Nicolet (Madison, Wis.) under the trade designation ALMEGA (#373500) was operated with the following conditions: 532 nm laser, 10-50% laser power, 25 micrometer pinhole aperture, 4.8-8.9 cm⁻¹ (672 lines/mm) resolution, 1.9 cm⁻¹ data spacing, 2 seconds exposure time, 16 exposures, and a 20× or 50×LWD objective.

Raman microscopical line mapping studies were performed utilizing a motorized x-y stage and z-axis focal control available from Prior (Rockland, Mass.) under the trade designation PROSCAN with software available from Thermo Nicolet, (Madison, Wis.) under the trade designation Atlus. The line maps were defined across the video image of the specimen, in 5 micrometer steps. A 50×long working distance (LWD) objective and 25 micrometer pinhole spectrograph aperture creates a spatial resolution of approximately 2 micrometers.

Point mapping studies were performed using a motorized x-y stage available from Prior (Rockland, Mass.) under the trade designation Proscan and auto-focusing capabilities of software available from Thermo Nicolet (Madion, Wis.) under the trade designation Atlus. Points to be analyzed were defined in Atlus software from the visual image; spectra were automatically collected using the spectral parameters described above.

Infrared microspectroscopy: Line mapping was performed using a fourier transform infrared (FTIR) spectrometer available under the trade designation NEXUS 670 (#374953) with an infrared (IR) microscope accessory with motorized x-y stage and z-axis focal control available under the trade designation CONTINUUM, all available from Thermo Nicolet (Madison, Wis.), with controlling software available under the trade designation ATLUS. The line maps were defined across the video image of the specimen, in 10 micrometer steps, using a 32×IR objective and a 15 micrometer reflex aperture setting. Spectra were collected at 4 cm⁻¹ spectral resolution in transmission mode, using an MCT-A detector with a 50 micrometer element. Samples were flattened onto a NaCl substrate.

Light Microscopical Comparisons

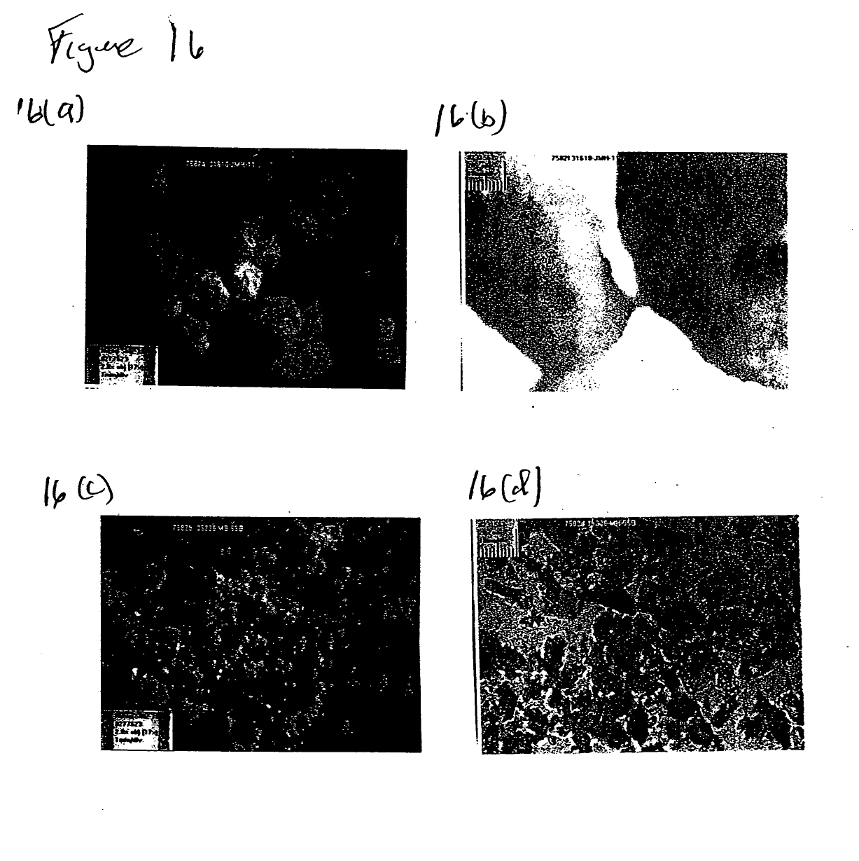

Microscopical examinations (7-400×) of the samples revealed significant differences in particle size and component distribution. Particle sizes of the sample produced by mortar and pestle were much smaller overall (FIGS. 16c and 16d) than the sample prepared by roller compacted granulation (FIGS. 16aand 16b).

The sample that was created via roller compaction of delavirdine mesylate and citric acid was composed of rounded/equant tan colored granules, typically 150-1000 micrometers in diameter. Upon crushing, the material appeared as a nearly uniform brown colored compacted mass, birefringent, yet with no detectable net extinction, indicating an agglomeration of crystalline material with domains in the micrometer (or less) size range. Individual particles of delavirdine mesylate and citric acid could not be recognized. Thus, individual components of this sample exist in large granules, but are closely associated (on a micrometer scale) within the structure of the granules.

The sample prepared by hand grinding delavirdine mesylate and citric acid, was a heterogeneous mixture of discrete particles in the 10-100 micrometer size domain, including tan-brown pleochroic (i.e. color varies with orientation) striated plates and colorless rounded/equant and plate shaped crystals, with 2^{nd}-3^{rd} order birefringence. The tan-brown colored particles were assumed to be delavirdine mesylate by virtue of their color. Thus, the individual components of this sample were much less closely associated in comparison to the sample prepared by roller compacted granulation.

Raman Microscopy

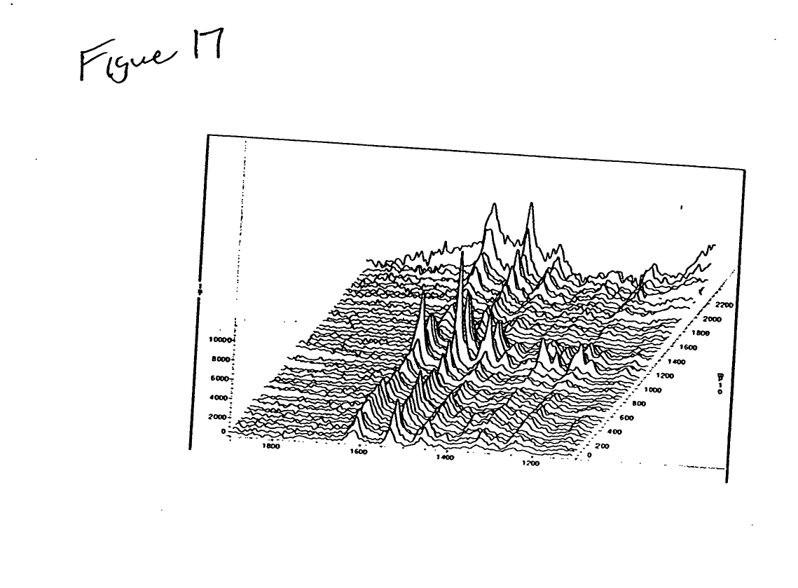

Heterogeneity assessments were provided using mapping capabilities of the Almega dispersive Raman microscope. For the sample prepared by roller compacted granulation, a granule was cross-sectioned, and a line map generated across the interior diameter, a distance of approximately 225 micrometers, in 5 micrometer steps. The Raman spectra obtained showed uniform features at all locations of the map, as shown in FIG. 17; although the peak intensities varied considerably across the granule, delavirdine mesylate features were evident in each location of the map, with no spectral features of citric acid evident. FIG. 18 shows a comparison of one point on the map to delavirdine mesylate and citric acid (hydrous).

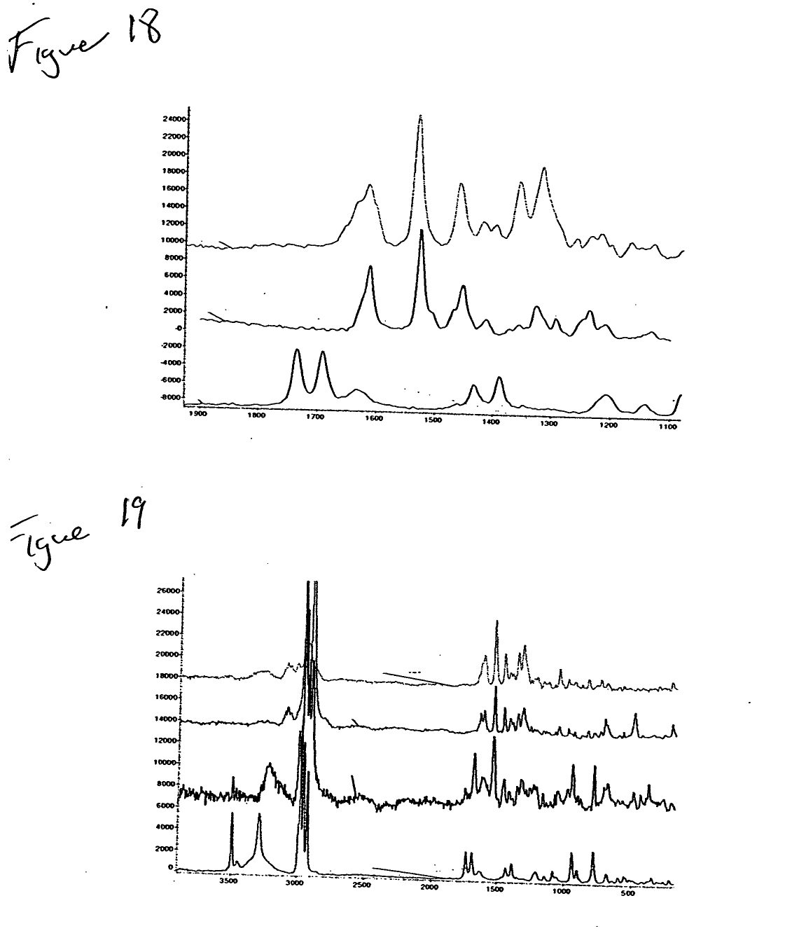

Individual particles in the sample produced by mortar and pestle were analyzed by Raman microscopy, which confirmed the heterogeneity observed via light microscopy. Pleochroic particles produced spectra similar to delavirdine mesylate, while colorless particles produced spectra with features of both delavirdine mesylate and citric acid features. Typical spectra are shown in FIG. 19.

Infrared Microspectroscopy

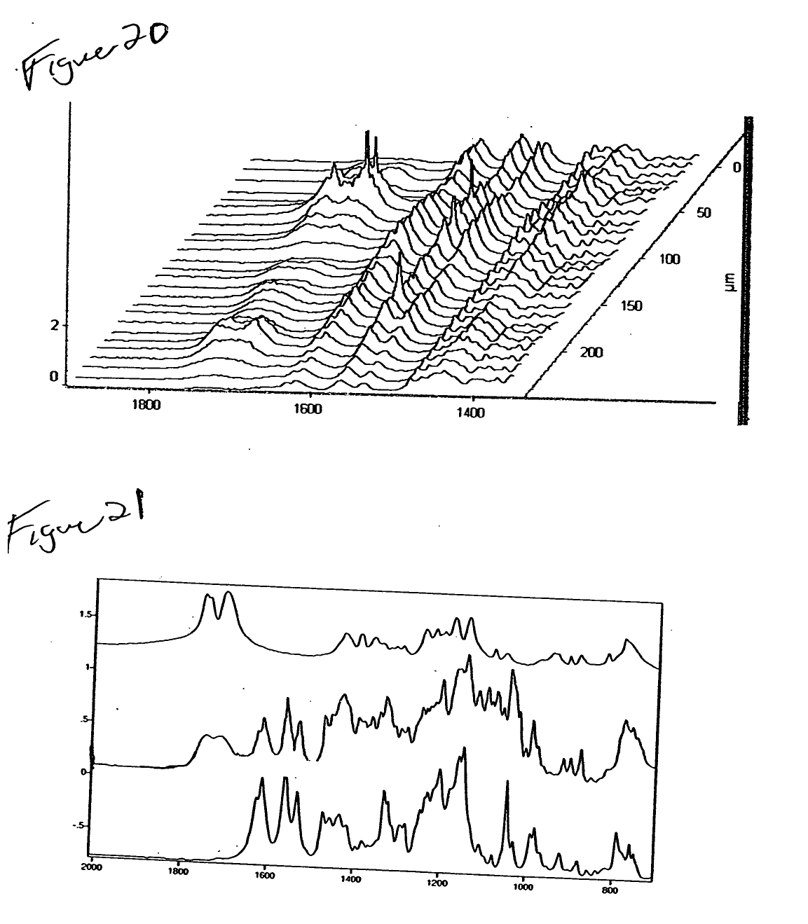

Since the relative Raman responses for delavirdine mesylate and citric acid were unknown, the sample produced by roller compacted granulation was further analyzed by IR microspectroscopy. A fragment of a granule was thinned to a few micrometers to allow transmission, then a line map generated at 15 micrometer spatial resolution. The line map across this preparation revealed the presence of delavirdine mesylate and citric acid features at all positions, confirming that the two components are blended to within the 15 micrometer spatial resolution of the technique. Variations in the relative peak heights were observed, which reflect variations in relative concentrations of delavirdine mesylate and citric acid on a micro scale. FIG. 20 shows spectra collected during the line scan; citric acid features are evident in the 1750-1700 cm⁻¹ region, while delavirdine mesylate is apparent in the 1650-1300 cm⁻¹ region. FIG. 21 shows a typical spectrum from the map against citric acid and delavirdine mesylate.

Conclusion

The microscopical evaluations revealed a significantly different particle size and component distribution in comparing roller compacted material to hand ground material. Roller compacted material consisted of large granules (150-1000 micrometers) that are tightly compacted, with uniformity of the mixture down to the spatial domains of the spectroscopical techniques (approximately 15 micrometers for IR). The hand ground material was primarily unassociated, discrete particles of the individual components, with blend uniformities on the order of approximately 100 micrometers.

### Example 10

### Dissolution Rate of a Co-compressed Mixture of a Poorly Soluble Non-ionizable Drug with a Solubilizing Excipient

Materials and Methods

The poorly soluble, non-ionizable drug illustrated in FIG. 1e can be prepared as described, for example, in PCT International Publication No. WO99/29688 (Poel et al.). Urea is a solubilizing excipient available from Aldrich Chemical Company, St. Louis, Mo.

Preparation of the Poorly Soluble, Non-ionizable Drug Illustrated in FIG. 1e Compressed Disks for Intrinsic Dissolution Rate Determination

The poorly soluble, non-ionizable drug illustrated in FIG. 1e and the poorly soluble, non-ionizable drug illustrated in FIG. 1e-urea-SDS (33:66:1 by weight) admixtures were weighed out and placed in a mortar and pestel. All three components were gently hand ground in the mortar and pestel for one minute. Pellets for the rotating disk experiment were prepared from about 20 mg of the mixed material and were co-compressed at 255 MPa (37,000 psi) in a manner similar to that described in Example 1.

Determination of the Intrinsic Dissolution Rate of the Poorly Soluble, Non-ionizable Drug Illustrated in FIG. 1e

The intrinsic dissolution rates of the poorly soluble, non-ionizable drug illustrated in FIG. 1e and the poorly soluble, non-ionizable drug illustrated in FIG. 1e-urea-SDS co-compressed admixtures were determined by a fiber optic automated rotating disk dissolution method in a manner similar to that described in Example 1. The dissolution media was 500 mL of 0.01N HCl at pH 2 at 37° C. The poorly soluble, non-ionizable drug illustrated in FIG. 1e was detected by monitoring the UV absorbance at 239.3 nm.

Results

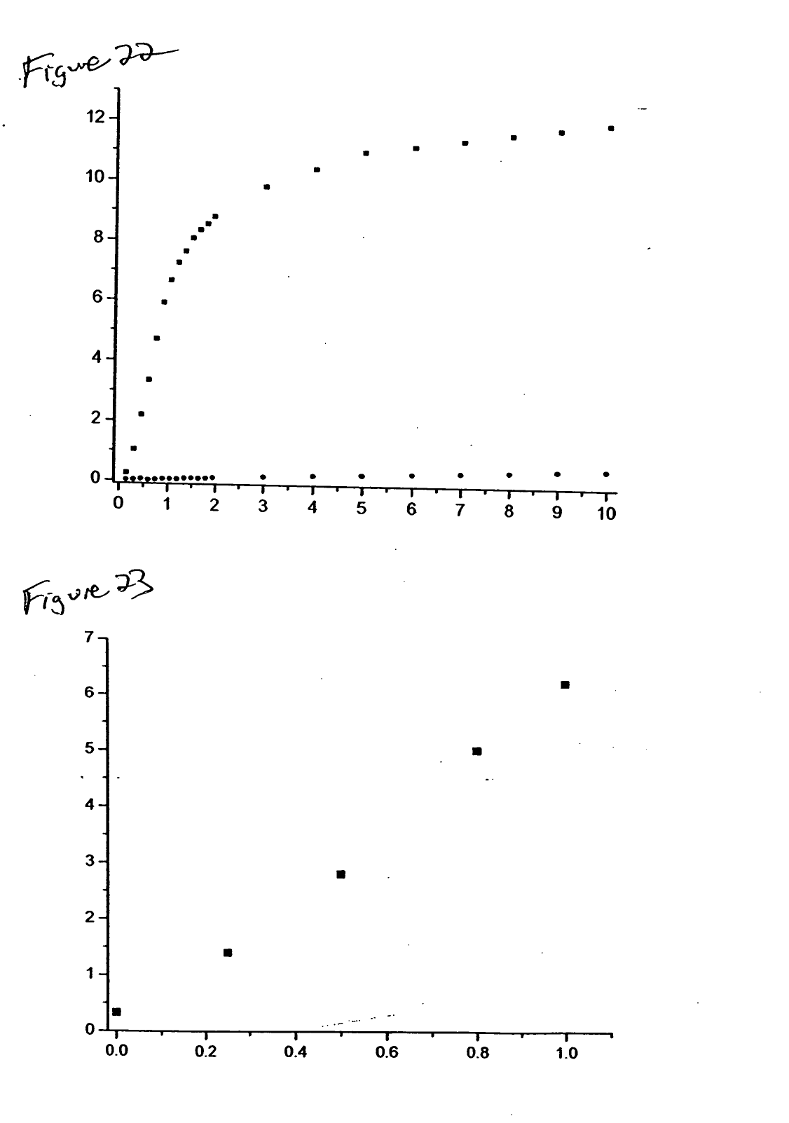

FIG. 22 shows the rotating disk dissolution results for the poorly soluble, non-ionizable drug illustrated in FIG. 1e alone (○) as compared to a co-compressed diffusion layer modulated solid made from 33% of the poorly soluble, non-ionizable drug illustrated in FIG. 1e, 66% urea, and 1% SDS ( [symbol] ). The co-compressed solid exhibited a large enhancement in the dissolution rate (calculated intrinsic dissolution rate=290 micrograms·sec⁻¹·cm⁻²) as compared to the bulk drug alone (calculated intrinsic dissolution rate=2.3 micrograms·sec⁻¹·cm⁻²). The initial slopes of the concentration versus time profiles showed that the co-compressed solid dissolved more than one hundred times faster than the bulk drug alone. This large enhancement in the dissolution rate resulted from the increased solubility of the poorly soluble, non-ionizable drug illustrated in FIG. 1e in the diffusion layer, which consisted of a concentrated solution of urea. Solubility data that has been collected showed that the solubility of the poorly soluble, non-ionizable drug illustrated in FIG. 1e increased significantly in urea solution (FIG. 23), and the dissolution rate for the diffusion layer modulated solid made from co-compressed urea and the poorly soluble, non-ionizable drug illustrated in FIG. 1e also showed improved dissolution.

### Example 11

### Dissolution of a (1:1) Co-compressed Admixture of a Soluble Salt of a Poorly Soluble, Acidic Drug and a Basic Excipient

Materials and Methods

The drug illustrated in FIG. 1(f) is a poorly soluble, acidic drug that can be prepared as described, for example, in Example 68 of U.S. Pat. No. 6,077,850 (Carter et al.). The drug is a poorly water-soluble free acid with a pKa of about three and an intrinsic solubility of less than 1 microgram/mL. Therefore, the molecule has poor water solubility in aqueous media of acidic pH.

Tris(hydroxymethyl)aminomethane (TRIS) is a basic excipient available from Aldrich, St. Louis, Mo. Other excipients used in formulations included MCC Coarse (154645), Fast Flo Lactose, Croscarmellose Sodium, NF Type A (128622), Colloidal Silicon Dioxide NF (112250), and Magnesium Stearate NF Powder, and were of standard grade and were used without modification.

Since the drug illustrated in FIG. 1(f) is a poorly water-soluble acid, it is relatively insoluble in the pH environment present in the stomach. Therefore, the tris(hydroxymethyl)aminomethane (or TRIS) salt of the drug was prepared to provide a water soluble alternative solid form of the drug.

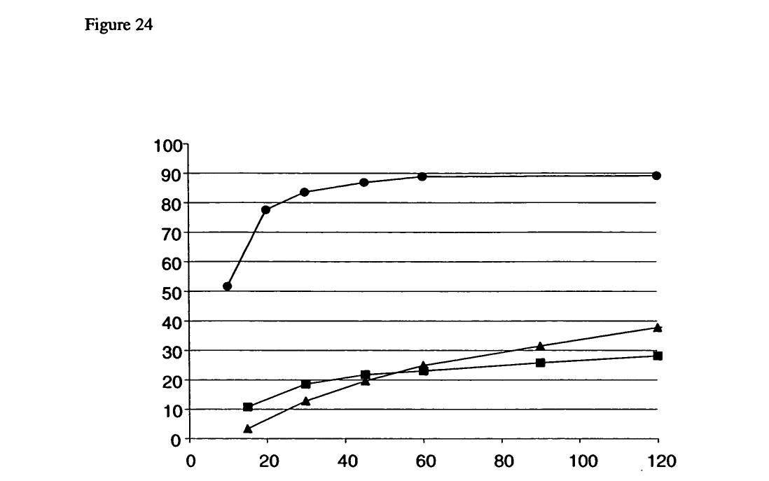

However, as shown in FIG. 24, the TRIS salt alone (formulated as bulk active pharmaceutical ingredient in a gelatin capsule) did not substantially enhance the dissolution rate of the drug. Since the TRIS salt has greater water solubility than the free acid, it might be expected to dissolve more rapidly. However, in pH 4.5 media, the free acid precipitated out from this formulation and formed large particles that dissolved more slowly than a capsule formulation made originally from the free acid.

It is important to note that precipitation of the free acid occurred, in this case, at a concentration where the free acid was undersaturated with respect to its bulk solubility at the pH of the dissolution experiment (pH 4.5). However, the concentration of the free acid in the diffusion layer was very high because of the relatively high water solubility of the salt, resulting in local precipitation in the diffusion layer.

To prevent precipitation of the free acid from the salt in the diffusion layer, a diffusion layer modulated solid was prepared. Since the drug was acidic, the basic excipient, TRIS, was used to raise the local pH to prevent precipitation. The pKa of TRIS is 8.1, so a concentrated solution of TRIS can raise the local pH in the diffusion layer significantly. The formulation composition was 1:1 mass ratio of the drug illustrated in FIG. 1(f) to TRIS and included: the TRIS salt of the drug illustrated in FIG. 1(f) (13.62 mg); TRIS (10.00 mg); MCC Coarse (154645) (35.19 mg); Fast Flo Lactose (35.19 mg); Croscarmellose Sodium, NF Type A (128622) (5.00 mg); Colloidal Silicon Dioxide NF (112250) (0.50 mg); and Magnesium Stearate NF Powder (0.50 mg).

The diffusion layer modulated solid was prepared using the following procedure. The TRIS salt of the drug illustrated in FIG. 1(f) was combined and mixed with additional TRIS. A disintegrant (e.g., croscarmellose) was added to the mixture and mixed well. The blend was then compressed into slugs using flat-face tooling and the Carver press. The slugs were ground up in a mortar and pestle and the ground granules were passed through a #20 mesh screen. Additional fillers (e.g., lactose), binders (e.g., microcrystalline cellulose), and disintegrant were added to the granules and mixed for an appropriate period of time. Lubricant (e.g., magnesium stearate) was then added and mixed for a short time. The final mixture was compressed into tablets on a Carver press using appropriate size tooling and compressional forces.

USP Dissolution Rate Determination

Dissolution profiles (illustrated in FIG. 24) were determined for the free acid of the poorly soluble, acidic drug illustrated in FIG. 1(f) in capsules (-▴-); for the TRIS salt of the poorly soluble, acidic drug illustrated in FIG. 1(f) (-▪-); and for the TRIS salt of the poorly soluble, acidic drug illustrated in FIG. 1(f)-TRIS (1:1) admixture co-compressed (Carver press) (-○-). Dissolution testing was completed on a USP type-II apparatus at 37° C. with a paddle speed of 50 revolutions per minute (rpm). Quantitation of the drug concentration was completed using high pressure liquid chromatography (HPLC) analysis. A pH 4.5 citrate buffer was used to control the PH during the dissolution experiment. The volume of the buffer was 900 mL. Dissolution tests were completed with 10 mg (free acid equivalent) formulations.

Results

FIG. 24 shows the results of the dissolution experiments for the co-compressed admixture. The co-compressed admixture showed a large enhancement in the dissolution rate and total amount dissolved as compared to the bulk salt alone. The enhanced dissolution may be due to prevention of the precipitation of free acid in the diffusion layer by the increased pH provided by TRIS solubilization around the drug salt/TRIS particles.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

2. The diffusion layer modulated solid of claim 1 wherein the weight ratio of the salt of the basic drug to the excipient is at least 15:85 and at most 95:5.

3. The diffusion layer modulated solid of claim 2 wherein the weight ratio of the salt of the basic drug to the excipient is at least 25:75 and at most 90:10.

4. The diffusion layer modulated solid of claim 3 wherein the weight ratio of the salt of the basic drug to the excipient is at least 35:65 and at most 85:15.

5. A composition comprising:
a diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method; and
a crystal growth inhibitor.

6. A diffusion layer modulated solid comprising particles comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof.

7. The diffusion layer modulated solid of claim 6 wherein the weight ratio of the salt of the basic drug to the excipient is at least 15:85 and at most 95:5.

8. The diffusion layer modulated solid of claim 6 wherein the average size of the particles is at least 1 micrometer.

9. The diffusion layer modulated solid of claim 8 wherein the average size of the particles is 5 micrometers to 400 micrometers.

10. The diffusion layer modulated solid of claim 6 wherein the particles form granules.

11. The diffusion layer modulated solid of claim 6 wherein the particles are homogeneous at a spatial domain of at most 15 micrometers.

12. A diffusion layer modulated solid preparable by a method comprising co-compressing at a pressure of at least 70 megapascals (10,000 pounds per square inch), a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof.

13. A diffusion layer modulated solid preparable by a method comprising spray drying a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof.

14. A capsule comprising a diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof, wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7.

15. The capsule of claim 14 further comprising a crystal growth inhibitor.

16. A tablet comprising a diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

17. The tablet of claim 16 further comprising a crystal growth inhibitor.

18. A method of preparing a diffusion layer modulated solid comprising preparing particles comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof.

19. The method of claim 18 wherein preparing the particles comprises:
roller compacting a mixture of the soluble salt of the basic drug and the excipient; and
granulating the compacted mixture to provide the particles.

20. The method of claim 19 wherein the roller compacting provides co-compression using at least 9000 newtons (2000 pounds force).

21. The method of claim 20 wherein the roller compacting provides co-compression using at least 18000 newtons (4000 pounds force).

22. The method of claim 21 wherein the roller compacting provides co-compression using at least 27000 newtons (6000 pounds force).

23. The method of claim 19 wherein the soluble salt of the basic drug comprises micronized particles before the roller compacting.

24. The method of claim 19 wherein the excipient comprises micronized particles before the roller compacting.

25. The method of claim 18 wherein preparing the particles comprises spray drying a mixture of the soluble salt of the basic drug and the excipient dissolved or dispersed in a volatile liquid.

26. The method of claim 25 wherein the volatile liquid comprises water.

27. A method of increasing the bioavailablity of a drug comprising providing a diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

28. A method of treating or preventing a disease comprising treating an animal with a diffusion layer modulated solid comprising a soluble salt of a basic drug having a solubility of at most 50 micrograms/ml in an aqueous fluid at pH 6 to pH 7 at 25° C. and an excipient selected from the group consisting of acidic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

29. A diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

30. The diffusion layer modulated solid of claim 29 wherein the weight ratio of the salt of the acidic drug to the excipient is at least 15:85 and at most 95:5.

31. The diffusion layer modulated solid of claim 30 wherein the weight ratio of the salt of the acidic drug to the excipient is at least 25:75 and at most 90:10.

32. The diffusion layer modulated solid of claim 31 wherein the weight ratio of the salt of the acidic drug to the excipient is at least 35:65 and at most 85:15.

33. A composition comprising:
a diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method; and
a crystal growth inhibitor.

34. A diffusion layer modulated solid comprising particles comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof.

35. The diffusion layer modulated solid of claim 34 wherein the weight ratio of the salt of the acidic drug to the excipient is at least 15:85 and at most 95:5.

36. The diffusion layer modulated solid of claim 34 wherein the average size of the particles is at least 1 micrometer.

37. The diffusion layer modulated solid of claim 36 wherein the average size of the particles is 5 micrometers to 400 micrometers.

38. The diffusion layer modulated solid of claim 34 wherein the particles form granules.

39. The diffusion layer modulated solid of claim 34 wherein the particles are homogeneous at a spatial domain of at most 15 micrometers.

40. A diffusion layer modulated solid preparable by a method comprising co-compressing at a pressure of at least 70 megapascals (10,000 pounds per square inch), a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof.

41. A diffusion layer modulated solid preparable by a method comprising spray drying a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof.

42. A capsule comprising a diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7.

43. The capsule of claim 42 further comprising a crystal growth inhibitor.

44. A tablet comprising a diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

45. The tablet of claim 44 further comprising a crystal growth inhibitor.

46. A method of preparing a diffusion layer modulated solid comprising preparing particles comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof.

47. The method of claim 46 wherein preparing the particles comprises:
roller compacting a mixture of the soluble salt of the acidic drug and the excipient; and
granulating the compacted mixture to provide the particles.

48. The method of claim 47 wherein the roller compacting provides co-compression using at least 9000 newtons (2000 pounds force).

49. The method of claim 48 wherein the roller compacting provides co-compression using at least 18000 newtons (4000 pounds force).

50. The method of claim 49 wherein the roller compacting provides co-compression using at least 27000 newtons (6000 pounds force).

51. The method of claim 47 wherein the soluble salt of the acidic drug comprises micronized particles before the roller compacting.

52. The method of claim 47 wherein the excipient comprises micronized particles before the roller compacting.

53. The method of claim 46 wherein preparing the particles comprises spray drying a mixture of the soluble salt of the acidic drug and the excipient dissolved or dispersed in a volatile liquid.

54. The method of claim 53 wherein the volatile liquid comprises water.

55. A method of increasing the bioavailablity of a drug comprising providing a diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

56. A method of treating or preventing a disease comprising treating an animal with a diffusion layer modulated solid comprising a soluble salt of an acidic drug having a solubility of at most 50 micrograms/ml in an aqueous fluid at pH 6 to pH 7 at 25° C. and an excipient selected from the group consisting of basic excipients, solubilizing excipients, and combinations thereof; wherein for at least one pH, the intrinsic dissolution rate of the diffusion layer modulated solid is at least 10% greater than the intrinsic dissolution rate of the drug salt alone at the same pH, and wherein the dissolution rates are both measured at 25° C. in water at a pH of 1 to 7 using a rotating disk method.

57. A diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7 using a rotating disk method.

58. The diffusion layer modulated solid of claim 57 wherein the weight ratio of the non-ionizable drug to the solubilizing excipient is at least 15:85 and at most 95:5.

59. The diffusion layer modulated solid of claim 58 wherein the weight ratio of the non-ionizable drug to the solubilizing excipient is at least 25:75 and at most 90:10.

60. The diffusion layer modulated solid of claim 59 wherein the weight ratio of the non-ionizable drug to the solubilizing excipient is at least 35:65 and at most 85:15.

61. A composition comprising:
a diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7 using a rotating disk method; and
a crystal growth inhibitor.

62. A diffusion layer modulated solid comprising particles comprising a non-ionizable drug having a solubility of at most 50 micrograms/ml in an aqueous fluid at pH 6 to pH 7 at 25° C. and a solubilizing excipient.

63. The diffusion layer modulated solid of claim 62 wherein the weight ratio of the non-ionizable drug to the solubilizing excipient is at least 15:85 and at most 95:5.

64. The diffusion layer modulated solid of claim 62 wherein the average size of the particles is at least 1 micrometer.

65. The diffusion layer modulated solid of claim 64 wherein the average size of the particles is 5 micrometers to 400 micrometers.

66. The diffusion layer modulated solid of claim 62 wherein the particles form granules.

67. The diffusion layer modulated solid of claim 62 wherein the particles are homogeneous at a spatial domain of at most 15 micrometers.

68. A diffusion layer modulated solid preparable by a method comprising co-compressing a non-ionizable drug having a solubility of at most 50 micrograms/ml in an aqueous fluid at pH 6 to pH 7 at 25° C. and a solubilizing excipient at a pressure of at least 70 megapascals (10,000 pounds per square inch).

69. A diffusion layer modulated solid preparable by a method comprising spray drying a non-ionizable drug having a solubility of at most 50 micrograms/ml in an aqueous fluid at pH 6 to pH 7 at 25° C. and a solubilizing excipient.

70. A capsule comprising a diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7.

71. The capsule of claim 70 further comprising a crystal growth inhibitor.

72. A tablet comprising a diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7 using a rotating disk method.

73. The tablet of claim 72 further comprising a crystal growth inhibitor.

74. A method of preparing a diffusion layer modulated solid comprising preparing particles comprising a non-ionizable drug having a solubility of at most 50 micrograms/ml in an aqueous fluid at pH 6 to pH 7 at 25° C. and a solubilizing excipient.

75. The method of claim 74 wherein preparing the particles comprises:
roller compacting a mixture of the non-ionizable drug and the solubilizing excipient; and
granulating the compacted mixture to provide the particles.

76. The method of claim 75 wherein the roller compacting provides co-compression using at least 9000 newtons (2000 pounds force).

77. The method of claim 76 wherein the roller compacting provides co-compression using at least 18000 newtons (4000 pounds force).

78. The method of claim 77 wherein the roller compacting provides co-compression using at least 27000 newtons (6000 pounds force).

79. The method of claim 75 wherein the non-ionizable drug comprises micronized particles before the roller compacting.

80. The method of claim 75 wherein the solubilizing excipient comprises micronized particles before the roller compacting.

81. The method of claim 74 wherein preparing the particles comprises spray drying a mixture of the non-ionizable drug and the solubilizing excipient dissolved or dispersed in a volatile liquid.

82. The method of claim 81 wherein the volatile liquid comprises water.

83. A method of increasing the bioavailablity of a drug comprising providing a diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7 using a rotating disk method.

84. A method of treating or preventing a disease comprising treating an animal with a diffusion layer modulated solid comprising a non-ionizable drug having a solubility of at most 50 micrograms/ml in an aqueous fluid at pH 6 to pH 7 at 25° C. and a solubilizing excipient, wherein for at least one pH, the intrinsic dissolution rate of the diffusion layer modulated solid is at least 10% greater than the intrinsic dissolution rate of the drug alone at the same pH, and wherein the dissolution rates are both measured at 25° C. in water at a pH of 1 to 7 using a rotating disk method.

85. A diffusion layer modulated solid comprising delavirdine mesylate and an acidic excipient, wherein for at least one pH, the intrinsic dissolution rate of the diffusion layer modulated solid is at least 10% greater than the intrinsic dissolution rate of delavirdine mesylate alone at the same pH, and wherein the dissolution rates are both measured at 25° C. in water at a pH of 1 to 7 using a rotating disk method.

86. A diffusion layer modulated solid comprising tipranavir disodium and a basic excipient, wherein for at least one pH, the intrinsic dissolution rate of the diffusion layer modulated solid is at least 10% greater than the intrinsic dissolution rate of tipranavir disodium alone at the same pH, and wherein the dissolution rates are both measured at 25° C. in water at a pH of 1 to 7 using a rotating disk method.

87. A diffusion layer modulated solid comprising the hydrochloride salt of the basic drug illustrated in FIG. 1c and an acidic excipient, wherein for at least one pH, the intrinsic dissolution rate of the diffusion layer modulated solid is at least 10% greater than the intrinsic dissolution rate of the hydrochloride salt of the basic drug illustrated in FIG. 1c alone at the same pH, and wherein the dissolution rates are both measured at 25° C. in water at a pH of 1 to 7 using a rotating disk method.

88. A diffusion layer modulated solid comprising the soluble hydrochloride salt illustrated in FIG. 1d and an acidic excipient, wherein for at least one pH, the intrinsic dissolution rate of the diffusion layer modulated solid is at least 10% greater than the intrinsic dissolution rate of the soluble hydrochloride salt illustrated in FIG. 1d alone at the same pH, and wherein the dissolution rates are both measured at 25° C. in water at a pH of 1 to 7 using a rotating disk method.

89. A diffusion layer modulated solid comprising the non-ionizable drug illustrated in FIG. 1e and a solubilizing excipient, wherein for at least one pH, the intrinsic dissolution rate of the diffusion layer modulated solid is at least 10% greater than the intrinsic dissolution rate of the non-ionizable drug illustrated in FIG. 1e alone at the same pH, and wherein the dissolution rates are both measured at 25° C. in water at a pH of 1 to 7 using a rotating disk method.
